(19) United States
(12) Patent Application Publication
(10) Pub. No.: US 2026/0248365 A1
(43) Pub. Date: Aug. 27, 2026

(54) DIAGNOSTIC APPARATUS, DIAGNOSTIC SYSTEM, DIAGNOSTIC METHOD, AND PROGRAM

(71) Applicant: FUJIFILM Corporation, Tokyo (JP)

(72) Inventors: Hiroyuki ISOBE, Kanagawa (JP); Kazunari TOYAMA, Kanagawa (JP)

(73) Assignee: FUJIFILM Corporation, Tokyo (JP)

(21) Appl. No.: 19/647,775

(22) Filed: Apr. 14, 2026

Related U.S. Application Data
(63) Continuation of application No. PCT/JP2024/033195, filed on Sep. 18, 2024.

(30) Foreign Application Priority Data
Oct. 16, 2023 (JP) 2023-178501

(51) Int. Cl. A61B 1/00; A61B 1/05; A61B 1/06

(52) U.S. Cl. CPC A61B 1/00057; A61B 1/000095; A61B 1/000096; A61B 1/05; A61B 1/0676

(57) ABSTRACT

A diagnostic apparatus includes a processor that is used for an endoscope having a distal end portion configured to irradiate an inside of a body with light, the distal end portion being provided with an optical system of a camera configured to image the inside of the body. The processor is configured to: execute a diagnosis of the optical system by performing image analysis using AI on a rear surface image obtained by imaging, by the camera, a rear surface in a state in which the rear surface is irradiated with the light in a situation in which a cap that is mounted on the distal end portion to cover the optical system, the cap having a rear surface configured to reflect the light, is mounted on the distal end portion The processor is configured to: output a diagnostic result obtained by executing the diagnosis.

### CROSS-REFERENCE TO RELATED APPLICATIONS

This application is a continuation application of International Application No. PCT/JP2024/033195, filed September 18, 2024, the disclosure of which is incorporated herein by reference in its entirety. Further, this application claims priority from Japanese Patent Application No. 2023-178501, filed October 16, 2023, the disclosure of which is incorporated herein by reference in its entirety.

### BACKGROUND

### 1. Technical Field

The present disclosure relates to a diagnostic apparatus, a diagnostic system, a diagnostic method, and a program.

### 2. Related Art

JP5162374B discloses a deviation amount measurement device for an endoscope image. The deviation amount measurement device for an endoscope image described in JP5162374B comprises a test chart on which a test pattern is drawn, a positioning means, an image synthesizing means, and a deviation amount acquisition unit. The positioning means positions any one of an insertion part distal end of an electronic endoscope to be inserted into a body cavity or the test chart with respect to the other. The image synthesizing means synthesizes a mask image, which is provided with an exposed portion that hides an invalid region of the endoscope image and exposes only a valid region in the endoscope image obtained by imaging the test chart with the electronic endoscope after the positioning by the positioning means, with a standard image having a reference pattern on the exposed portion. The deviation amount acquisition unit acquires a deviation amount of the test pattern with respect to the reference pattern from a composite image synthesized by the image synthesizing means.

JP2021-182950A discloses an information processing apparatus comprising an evaluation unit that evaluates a state of a medical instrument based on a sound signal of a sound generated by the medical instrument. The evaluation unit compares the sound signal with a past sound signal of the medical instrument and evaluates the state of the medical instrument based on a result of the comparison. The evaluation unit evaluates the state of the medical instrument and detects or predicts a failure of the medical instrument. In addition, JP2021-182950A discloses that a determination of the failure of the medical instrument is executed by a cloud.

### SUMMARY

One embodiment according to the present disclosure provides a diagnostic apparatus, a diagnostic system, a diagnostic method, and a program capable of accurately diagnosing an optical system provided in an endoscope.

A first aspect according to the present disclosure is a diagnostic apparatus including a processor that is used for an endoscope having a distal end portion configured to irradiate an inside of a body with light, the distal end portion being provided with an optical system of a camera configured to image the inside of the body, in which the processor is configured to: execute a diagnosis of the optical system by performing image analysis using AI on a rear surface image obtained by imaging, by the camera, a rear surface in a state in which the rear surface is irradiated with the light in a situation in which a cap that is mounted on the distal end portion to cover the optical system, the cap having a rear surface configured to reflect the light, is mounted on the distal end portion; and output a diagnostic result obtained by executing the diagnosis.

A second aspect according to the present disclosure is the diagnostic apparatus according to the first aspect, in which the diagnosis includes defect presence and absence identification processing of identifying presence or absence of a defect of the optical system.

A third aspect according to the present disclosure is the diagnostic apparatus according to the first or second aspect, in which the diagnosis includes type identification processing of identifying a type of a defect of the optical system, and the type of the defect includes a first type that does not require collection of the optical system and a second type that requires the collection of the optical system.

A fourth aspect according to the present disclosure is the diagnostic apparatus according to the third aspect, in which the type identification processing includes a classification processing of classifying the type of the defect into the first type and the second type.

A fifth aspect according to the present disclosure is the diagnostic apparatus according to the third or fourth aspect, in which the first type is contamination of the optical system, and the second type is a failure of the optical system.

A sixth aspect according to the present disclosure is the diagnostic apparatus according to any one of the first to fifth aspects, in which the image analysis is implemented by inputting the rear surface image to a trained model that generates information assuming the diagnostic result or information serving as a basis for a diagnosis result by inputting information assuming the rear surface image, to generate the diagnosis result or the information serving as the basis for the diagnosis result.

A seventh aspect according to the present disclosure is the diagnostic apparatus according to any one of the third to fifth aspects, in which the AI includes a first type identification AI that identifies the first type based on the rear surface image, and a second type identification AI that identifies the second type based on the rear surface image.

An eighth aspect according to the present disclosure is the diagnostic apparatus according to any one of the first to seventh aspects, in which the AI is enhanced by performing retraining based on the diagnostic result.

A ninth aspect according to the present disclosure is the diagnostic apparatus according to any one of the first to eighth aspects, in which the diagnosis includes position identification processing of identifying a position at which a defect of the optical system occurs.

A tenth aspect according to the present disclosure is the diagnostic apparatus according to any one of the first to ninth aspects, in which the rear surface image is an image obtained by performing noise removal processing and/or edge extraction processing.

An eleventh aspect according to the present disclosure is the diagnostic apparatus according to the tenth aspect, in which the rear surface image is an image obtained by performing the edge extraction processing after performing the noise removal processing.

A twelfth aspect according to the present disclosure is the diagnostic apparatus according to any one of the first to eleventh aspects, in which the rear surface has a first region that reflects the light and a second region that has a lower reflectance than the first region.

A thirteenth aspect according to the present disclosure is the diagnostic apparatus according to the twelfth aspect, in which an area of the first region is larger than an area of the second region, and a spatial frequency of the second region is higher than a spatial frequency of the first region.

A fourteenth aspect according to the present disclosure is the diagnostic apparatus according to the twelfth or thirteenth aspect, in which the second region is a test chart.

A fifteenth aspect according to the present disclosure is the diagnostic apparatus according to any one of the first to fourteenth aspects, in which the rear surface is formed in a curved shape.

A sixteenth aspect according to the present disclosure is the diagnostic apparatus according to any one of the first to fifteenth aspects, in which the rear surface is a surface that diffuses the light.

A seventeenth aspect according to the present disclosure is the diagnostic apparatus according to any one of the first to sixteenth aspects, in which the cap is formed in a two-layer structure.

An eighteenth aspect according to the present disclosure is the diagnostic apparatus according to the seventeenth aspect, in which the two-layer structure is formed of an inner layer and an outer layer, and a hollow region is provided between the inner layer and the outer layer.

A nineteenth aspect according to the present disclosure is the diagnostic apparatus according to any one of the first to eighteenth aspects, in which a color of an outer surface of the cap is black.

A twentieth aspect according to the present disclosure is the diagnostic apparatus according to any one of the first to nineteenth aspects, in which the cap is mounted on the distal end portion via an attachment, and a diameter of a portion of the attachment that connects the cap and the distal end portion is adjustable.

A twenty-first aspect according to the present disclosure is a diagnostic system including: a terminal that is used for an endoscope having a distal end portion configured to irradiate an inside of a body with light, the distal end portion being provided with an optical system of a camera configured to image an inside of the body; and a server, in which the terminal is configured to transmit, to the server, a rear surface image obtained by imaging, by the camera, a rear surface in a state in which the rear surface is irradiated with the light in a situation in which a cap that is mounted on the distal end portion to cover the optical system, the cap having a rear surface configured to reflect the light is mounted on the distal end portion, the server is configured to: execute a diagnosis of the optical system by performing image analysis using AI on a rear surface image; and transmit a diagnosis result obtained by executing the diagnosis to the terminal, and the terminal receives the diagnostic result.

A twenty-second aspect according to the present disclosure is a diagnostic method that is used for an endoscope having a distal end portion configured to irradiate an inside of a body with light, the distal end portion being provided with an optical system of a camera configured to image the inside of the body, the diagnostic method including: executing a diagnosis of the optical system by performing image analysis using AI on a rear surface image obtained by imaging, by the camera, a rear surface in a state in which the rear surface is irradiated with the light in a situation in which a cap that is mounted on the distal end portion to cover the optical system, the cap having a rear surface configured to reflect the light is mounted on the distal end portion; and outputting a diagnostic result obtained by executing the diagnosis.

A twenty-third aspect according to the present disclosure is a program causing a computer that is used for an endoscope having a distal end portion configured to irradiate an inside of a body with light and having a distal end portion provided with an optical system of a camera configured to image the inside of the body to execute a process including: executing a diagnosis of the optical system by performing image analysis using AI on a rear surface image obtained by imaging, by the camera, a rear surface in a state in which the rear surface is irradiated with the light in a situation in which a cap that is mounted on the distal end portion to cover the optical system, the cap having a rear surface configured to reflect the light is mounted on the distal end portion; and outputting a diagnostic result obtained by executing the diagnosis.

### BRIEF DESCRIPTION OF THE DRAWINGS

Exemplary embodiments according to the technique of the present disclosure will be described in detail based on the following figures, wherein:

FIG. 1 is a conceptual diagram showing an example of an aspect in which an endoscope apparatus included in a diagnostic system is used by a doctor;

FIG. 2 is a conceptual diagram showing an example of an overall configuration of the diagnostic system;

FIG. 3 is a block diagram showing an example of a hardware configuration of an electric system of the diagnostic system;

FIG. 4 is a screen diagram showing an example of an aspect in which a frame generated by imaging an inside of a large intestine with a camera is displayed in a first display region;

FIG. 5 is a conceptual diagram showing a configuration example of a cap and an attachment;

FIG. 6 is a conceptual diagram showing an example of an aspect in which a camera images a rear surface of a cap in a state in which the cap is mounted on a distal end portion of an endoscope via an attachment;

FIG. 7 is a block diagram illustrating an example of functions of main units of a processor included in a medical support device and an example of information stored in a storage;

FIG. 8 is a conceptual diagram showing an example of processing contents of the medical support device and a processing apparatus;

FIG. 9 is a conceptual diagram showing an example of processing contents of a controller and an example of a content displayed on a screen of a display device;

FIG. 10 is a flowchart showing an example of a flow of a diagnosis process;

FIG. 11 is a conceptual diagram showing a first modification example of the processing content of the processing apparatus;

FIG. 12 is a conceptual diagram showing a second modification example of the processing content of the processing apparatus;

FIG. 13 is a conceptual diagram showing a third modification example of the processing content of the processing apparatus;

FIG. 14 is a conceptual diagram showing a fourth modification example of the processing content of the processing apparatus; and

FIG. 15 is a conceptual diagram showing an example of an aspect in which an execution unit executes noise removal processing, edge extraction processing, recognition processing, comprehensive determination processing, and/or generation processing.

### DETAILED DESCRIPTION

Hereinafter, an example of embodiments of a diagnostic apparatus, a diagnostic system, a diagnostic method, and a program according to the present disclosure will be described with reference to the accompanying drawings.

First, the terms used hereinafter will be described.

CPU is an abbreviation for "central processing unit". GPU is an abbreviation for "Graphics Processing Unit". GPGPU is an abbreviation for “general-purpose computing on graphics processing units”. APU is an abbreviation for “accelerated processing unit”. TPU is an abbreviation for "tensor processing unit". RAM is an abbreviation for "Random Access Memory". NVM refers to an abbreviation for "Non-Volatile Memory". EEPROM is an abbreviation for "electrically erasable programmable read-only memory". ASIC is an abbreviation for "application specific integrated circuit". PLD is an abbreviation for "programmable logic device". FPGA indicates the abbreviation for “field-programmable gate array”. SoC refers to an abbreviation of “system-on-a-chip”. SSD is an abbreviation for "solid state drive". USB is an abbreviation for "universal serial bus". HDD refers to an abbreviation of “hard disk drive”. EL is an abbreviation for "electro-luminescence". CMOS is an abbreviation for "complementary metal oxide semiconductor". CCD is an abbreviation for "charge coupled device". AI refers to an abbreviation for "Artificial Intelligence". BLI is an abbreviation for "blue light imaging". LCI is an abbreviation for "linked color imaging". I/F refers to an abbreviation of an "Interface". SSL stands for “Sessile Serrated Lesion”. LAN refers to an abbreviation of a "Local Area Network". WAN is an abbreviation of "Wide Area Network". 5G is an abbreviation for “5th generation mobile communication system”. IC refers to an abbreviation for "Integrated Circuit".

Hereinafter, a processor with a reference numeral (hereinafter, simply referred to as "processor") may be one physical or virtual computing device or a combination of a plurality of physical or virtual computing devices. Furthermore, the processor may be one type of computing device or may be a combination of a plurality of types of computing devices. Examples of the operation device include a CPU, a GPU, a GPGPU, an APU, or a TPU.

In the following description, a memory with a reference numeral is a memory, such as a RAM, temporarily storing information and is used as a work memory by the processor.

In the following description, a storage with a reference numeral is one or a plurality of non-volatile storage devices that store various programs, various parameters, and the like. Examples of the non-volatile storage device include a flash memory, a magnetic disk, and a magnetic tape. In addition, another example of the storage is a cloud storage.

In the following embodiment, an external I/F with a reference numeral controls the transmission and reception of various types of information among a plurality of devices connected to each other. An example of the external I/F is a USB interface. A communication I/F including a communication processor, an antenna, and the like may be applied to the external I/F. The communication I/F controls communication between a plurality of computers. Examples of a communication standard applied to the communication I/F include a wireless communication standard including 5G, Wi-Fi (registered trademark), and Bluetooth (registered trademark).

In the following embodiment, "A and/or B" is synonymous with "at least one of A or B". That is, “A and/or B” means “A, B, or a combination of A and B”. In addition, in the present specification, the same concept as in the case of “A and/or B” applies to a case where three or more matters are expressed together by “and/or”.

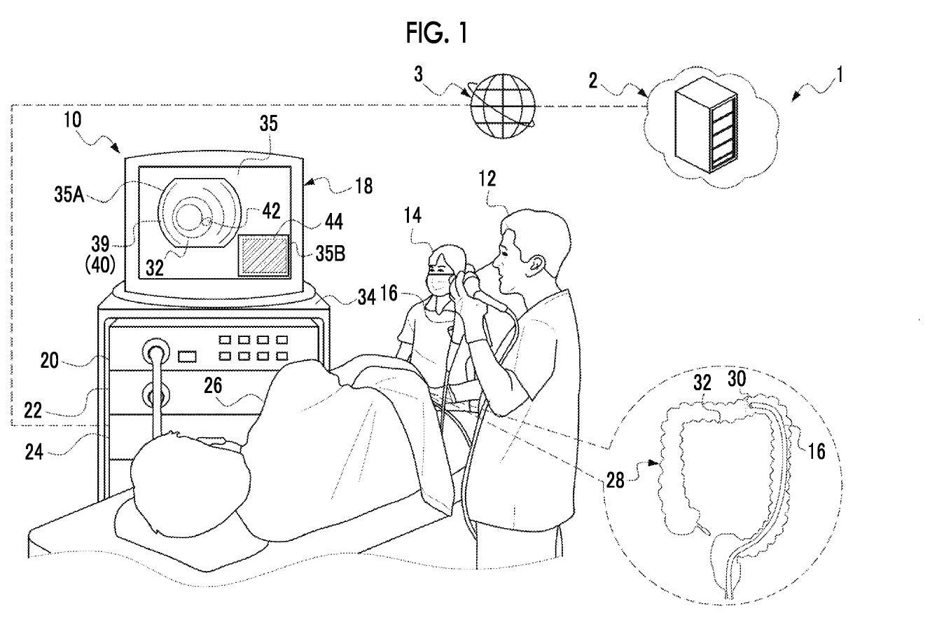

FIG. 1 is a conceptual diagram showing an example of an aspect in which a diagnostic system 1 is used. As shown in FIG. 1, the diagnostic system 1 comprises a processing device 2 and an endoscope apparatus 10, and the processing device 2 and the endoscope apparatus 10 are connected to be communicable with each other via a network 3. Examples of the network 3 include the Internet. However, the Internet is merely an example, and examples of the other network 3 include a WAN and/or a LAN.

In the present embodiment, the diagnostic system 1 is an example of a "diagnostic system" according to the present disclosure, the processing device 2 is an example of a "server" according to the present disclosure, and the endoscope apparatus 10 is an example of a "diagnostic apparatus" and a "terminal" according to the present disclosure.

The endoscope apparatus 10 is used by a doctor 12 in endoscopy and the like. The endoscopy is assisted by a staff member such as a nurse 14.

Information obtained by the endoscope apparatus 10 is transmitted to the processing device 2 via the network 3. Examples of the processing device 2 include a cloud server. However, the cloud server is merely an example, and the processing device 2 may be an on-premises server or a personal computer. The processing device 2 receives the information transmitted from the endoscope apparatus 10, executes processing using the received information, and transmits a processing result obtained by executing the processing to the endoscope apparatus 10.

The endoscope apparatus 10 comprises an endoscope 16, a display device 18, a light source device 20, a control device 22, and a medical support device 24. In addition, the endoscope 16 in the present embodiment is an example of an "endoscope" according to the present disclosure.

The endoscope apparatus 10 is a modality for performing medical care on a large intestine 28 included in a body of a subject 26 (for example, a patient) by using the endoscope 16. In the present embodiment, the large intestine 28 is a target to be observed by the doctor 12.

The endoscope 16 is used by the doctor 12 and is inserted into the body of the subject 26. In the present embodiment, the endoscope 16 is inserted into the large intestine 28 that is a lumen organ of the subject 26.

The endoscope apparatus 10 causes the endoscope 16 inserted into the large intestine 28 of the subject 26 to image an inside of the large intestine 28 of the subject 26 and performs various medical treatments on the large intestine 28 as necessary.

The endoscope apparatus 10 acquires and outputs an image showing an aspect in the large intestine 28 by imaging the inside of the large intestine 28 of the subject 26. In the present embodiment, the endoscope apparatus 10 is an endoscope having an optical imaging function of capturing reflected light obtained by emitting light 30 inside the large intestine 28 and reflecting the light 30 from an intestinal wall 32 of the large intestine 28. The light 30 is an example of "light" according to the present disclosure.

It should be noted that, here, the endoscopy of the large intestine 28 has been described as an example, but this is merely an example, and the present disclosure is applicable to an endoscopy of a luminal organ, such as an esophagus, a stomach, a duodenum, or a trachea.

The light source device 20, the control device 22, and the medical support device 24 are installed in a wagon 34. In the wagon 34, a plurality of tables are provided along an up-down direction, and the medical support device 24, the control device 22, and the light source device 20 are installed from a lower table to an upper table. The display device 18 is installed on an uppermost table in the wagon 34.

The control device 22 controls the entire endoscope apparatus 10. The medical support device 24 performs various types of image processing on the image obtained by imaging the intestinal wall 32 with the endoscope 16 under the control of the control device 22. In addition, the medical support device 24 is communicably connected to the processing device 2 via the network 3, and requests the processing device 2 to provide a service, thereby receiving the requested service from the processing device 2.

The display device 18 displays various types of information including the image. Examples of the display device 18 include a liquid crystal display or an EL display. Furthermore, a tablet terminal equipped with a display may be used instead of the display device 18 or together with the display device 18.

A screen 35 is displayed on the display device 18. The screen 35 includes a plurality of display regions. The plurality of display regions are arranged side by side in the screen 35. In the example shown in FIG. 1, a first display region 35A and a second display region 35B are shown as examples of the plurality of display regions. A size of the first display region 35A is larger than a size of the second display region 35B. The first display region 35A is used as a main display region, and the second display region 35B is used as a sub-display region. A size relationship between the first display region 35A and the second display region 35B is not limited to this and may be any size relationship that falls within the screen 35.

An endoscopic video image 39 is displayed in the first display region 35A. The endoscopic video image 39 is a moving image acquired by imaging the intestinal wall 32 with the endoscope 16 inside the large intestine 28 of the subject 26. In the example shown in FIG. 1, a video image in which the intestinal wall 32 is captured is shown as an example of the endoscopic video image 39.

The intestinal wall 32 shown in the endoscopic video image 39 includes a lesion 42 (for example, in the example shown in FIG. 1, one lesion 42) as a region of interest (that is, an observation target region) focused on by the doctor 12, and the doctor 12 can visually recognize an aspect of the intestinal wall 32, including the lesion 42, through the endoscopic video image 39.

There are various types of the lesion 42, and examples of the types of the lesion 42 include a neoplastic polyp and a non-neoplastic polyp. Examples of the type of the neoplastic polyp include an adenomatous polyp (for example, SSL). Examples of the types of the non-neoplastic polyp include a hamartomatous polyp, a hyperplastic polyp, and an inflammatory polyp. In addition, the types shown here are types assumed in advance as the types of the lesion 42 in a case in which the endoscopy is performed on the large intestine 28, and the types of the lesion 42 may be different depending on the organ on which the endoscopy is performed.

In the present embodiment, for convenience of description, a form example is described in which one lesion 42 is shown in the endoscopic video image 39, but the present disclosure is not limited to this. The present disclosure is established even in a case where a plurality of the lesions 42 are shown in the endoscopic video image 39.

In the present embodiment, the lesion 42 is shown, but this is merely an example, and the region of interest (that is, the observation target region) focused on by the doctor 12 may be a feature region having some unique feature, such as an organ (for example, a duodenal papilla), a mark, an artificial treatment tool (for example, an artificial clip), a treated region (for example, a region in which a trace of removal of a polyp or the like remains), or the like.

The image displayed in the first display region 35A is one frame 40 included in a video image configured to include a plurality of frames 40 arranged in time series. That is, a plurality of frames 40 along the time series are displayed in the first display region 35A at a predetermined frame rate (for example, several tens of frames/second).

An example of the video image displayed in the first display region 35A is a video image in a live view mode. The live view method is only an example, and a post view method in which a moving image is temporarily stored in a memory or the like and then displayed may be employed. In addition, each frame included in a video image for recording stored in the memory or the like may be reproduced and displayed as the endoscopic video image 39 on the screen 35 (for example, in the first display region 35A).

In the screen 35, the second display region 35B is adjacent to the first display region 35A and is displayed in the lower right in the screen 35 in front view. A display position of the second display region 35B may be anywhere in the screen 35 of the display device 18. However, the display position is preferably displayed at a position comparable to the endoscopic video image 39.

In the second display region 35B, auxiliary information 44 that assists a medical determination by the doctor 12 or the like in the endoscopy, a determination by the doctor 12 or the like for a malfunction of the endoscope 16 in the endoscope maintenance, and the like is displayed. The auxiliary information 44 is information referred to by the doctor 12 or the like. Examples of the auxiliary information 44 include various types of information related to the subject 26 into which the endoscope 16 is inserted, and/or various types of information obtained by performing processing of diagnosing the endoscope 16 (for example, processing of determining whether or not a malfunction has occurred in the endoscope 16 or processing of specifying a malfunction that has occurred).

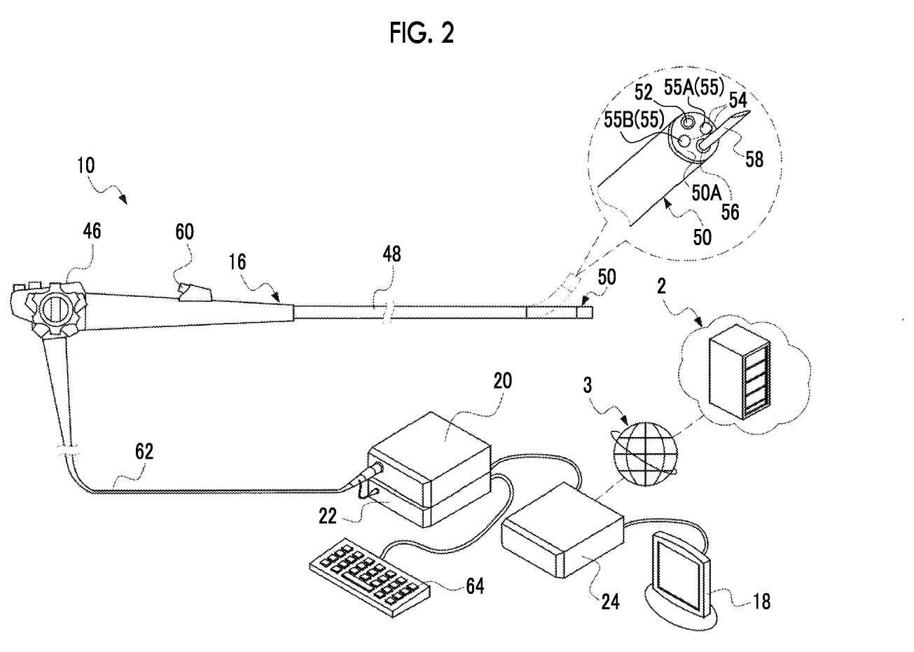

FIG. 2 is a conceptual diagram illustrating an example of an overall configuration of the endoscope apparatus 10. As illustrated in FIG. 2, the endoscope 16 comprises an operation part 46 and an insertion part 48. The insertion part 48 is partially curved by the operation of the operation part 46. The insertion part 48 is inserted into the large intestine 28 while being curved along the shape of the large intestine 28 (see FIG. 1) in accordance with the operation of the operation part 46 by the doctor 12 (see FIG. 1).

A distal end portion 50 of the insertion part 48 is provided with a camera 52, an illumination device 54, and a treatment tool opening 56. The camera 52 and the illumination device 54 are provided on a distal end surface 50A of the distal end portion 50. In addition, here, the form in which the camera 52 and the illumination device 54 are provided on the distal end surface 50A of the distal end portion 50 is given as an example. However, this is only an example. The camera 52 and the illumination device 54 may be provided on a side surface of the distal end portion 50 such that the endoscope 16 is configured as a side-viewing endoscope.

The camera 52 is mounted on the endoscope 16 and is inserted into a body cavity of the subject 26 to image the observation target region to generate the frame 40 as an endoscopic image. In the present embodiment, the camera 52 generates the endoscopic video image 39 including the plurality of frames 40 along the time series by imaging the inside of the body (for example, the inside of the large intestine 28) of the subject 26. An example of the camera 52 is a CMOS camera. However, this is only an example, and the camera 52 may be other types of cameras such as CCD cameras. In the present embodiment, the distal end portion 50 is an example of a "distal end portion" according to the present disclosure, and the camera 52 is an example of a "camera" according to the present disclosure.

The illumination device 54 includes an optical system 55. The optical system 55 includes illumination lenses 55A and 55B. The illumination lenses 55A and 55B are lenses including an objective lens, and the objective lens is exposed to the outside from the distal end surface 50A. The optical system 55 is an example of an "optical system" according to the present disclosure.

The illumination device 54 irradiates the light 30 (see FIG. 1) through the optical system 55. Examples of the types of the light 30 emitted from the illumination device 54 include visible light (for example, white light) and invisible light (for example, near-infrared light). In addition, the illumination device 54 irradiates special light through the optical system 55. Examples of the special light include light for BLI and/or light for LCI. The camera 52 images the inside of the large intestine 28 by using an optical method in a state in which the illumination device 54 irradiates the inside of the large intestine 28 with the light 30.

The treatment tool opening 56 is an opening through which a treatment tool 58 protrudes from the distal end portion 50. Further, the treatment tool opening 56 is also used as a suction port for suctioning blood, internal contaminants, and the like and a sending-out port for sending out fluid.

A treatment tool insertion opening 60 is formed in the operation part 46, and the treatment tool 58 is inserted into the insertion part 48 through the treatment tool insertion opening 60. The treatment tool 58 passes through the insertion part 48 and protrudes from the treatment tool opening 56 to the outside. In the example shown in FIG. 2, an aspect is shown in which a biopsy needle protrudes through the treatment tool opening 56 as the treatment tool 58. Here, the puncture needle is given as an example of the treatment tool 58. However, this is only an example. The treatment tool 58 may be grasping forceps, a papillotomy knife, a snare, a catheter, a guide wire, a cannula, and/or a puncture needle with a guide sheath.

The endoscope 16 is connected to the light source device 20 and the control device 22 through a universal cord 62. The medical support device 24 and a reception device 64 are connected to the control device 22. In addition to the processing device 2, the display device 18 is also connected to the medical support device 24. That is, the control device 22 is connected to the processing device 2 and the display device 18 via the medical support device 24.

Here, since the medical support device 24 is illustrated as an externally connected device for expanding a function performed by the control device 22, a form example is described in which the control device 22 and the display device 18 are indirectly connected to each other via the medical support device 24, but this is merely an example. For example, the display device 18 may be directly connected to the control device 22. In this case, for example, functions of the medical support device 24 may be mounted on the control device 22.

The reception device 64 receives an instruction from the doctor 12 and outputs the received instruction as an electric signal to the control device 22. Examples of the reception device 64 include a keyboard, a mouse, a touch panel, a foot switch, a microphone, and/or a remote operation device.

The control device 22 controls the light source device 20, transmits and receives various signals to and from the camera 52, or transmits and receives various signals to and from the medical support device 24.

The light source device 20 emits light under the control of the control device 22 to supply the light to the illumination device 54. A light guide is built in the illumination device 54, and the light supplied from the light source device 20 is emitted from the illumination lenses 55A and 55B via the light guide. The light emitted from the illumination lenses 55A and 55B is the light 30 (see FIG. 1). The control device 22 causes the camera 52 to perform imaging, acquires the endoscopic video image 39 (see FIG. 1) from the camera 52, and outputs the endoscopic video image 39 to a predetermined output destination (for example, the medical support device 24).

The medical support device 24 executes various types of image processing on the endoscopic video image 39 input from the control device 22 to support the medical treatment (here, for example, endoscopy). The medical support device 24 outputs the endoscopic video image 39 subjected to various types of image processing to a predetermined output destination (for example, the display device 18).

Here, the form example has been described in which the endoscopic video image 39 output from the control device 22 is output to the display device 18 via the medical support device 24, but this is merely an example. For example, the control device 22 and the display device 18 may be connected to each other, and the endoscopic video image 39 that has been subjected to the image processing by the medical support device 24 may be displayed on the display device 18 via the control device 22.

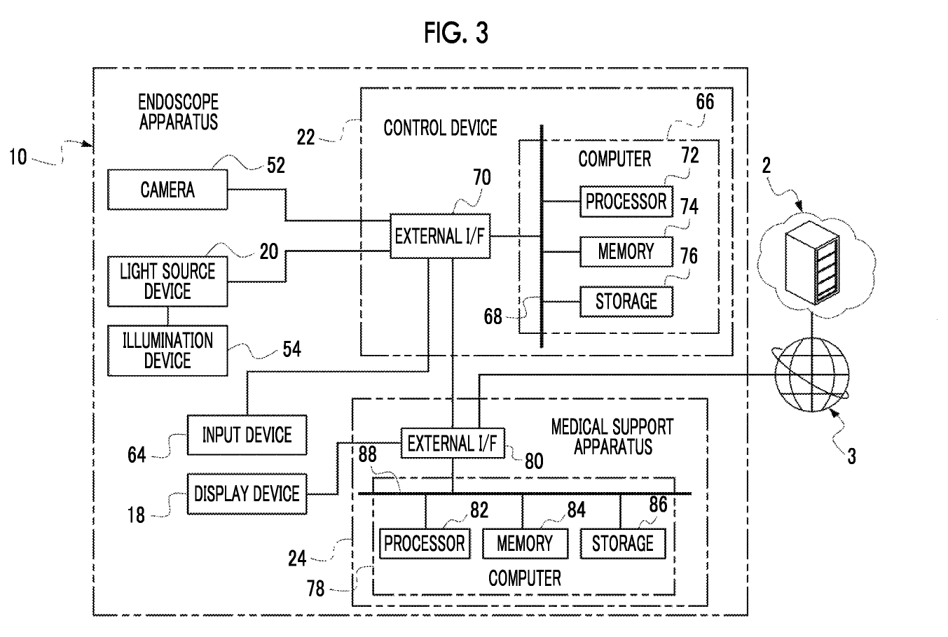

FIG. 3 is a block diagram showing an example of a hardware configuration of an electric system of the endoscope apparatus 10. As illustrated in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 controls the transmission and the reception of various types of information between one or more devices (hereinafter also referred to as "first external devices") present outside the control device 22 and the processor 72.

As one of the first external devices, the camera 52 is connected to the external I/F 70, and the external I/F 70 transmits and receives various types of information between the camera 52 and the processor 72. The processor 72 controls the camera 52 via the external I/F 70. In addition, the processor 72 acquires the endoscopic video image 39 (see FIG. 1) obtained by imaging the inside of the large intestine 28 (see FIG. 1) with the camera 52 via the external I/F 70.

As one of the first external devices, the light source device 20 is connected to the external I/F 70, and the external I/F 70 transmits and receives various types of information between the light source device 20 and the processor 72. The light source device 20 supplies the light to the illumination device 54 under the control of the processor 72. The illumination device 54 performs irradiation with the light supplied from the light source device 20.

The reception device 64 is connected to the external I/F 70 as one of the first external devices, and the processor 72 acquires the instruction received by the reception device 64 via the external I/F 70 and executes processing in accordance with the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the present embodiment, the computer 78 is an example of a "computer" according to the present disclosure, and the processor 82 is an example of a "processor" according to the present disclosure.

It should be noted that a hardware configuration (that is, the processor 82, the memory 84, and the storage 86) of the computer 78 is essentially the same as the hardware configuration of the computer 66, and thus the description of the hardware configuration of the computer 78 will be omitted here.

The external I/F 80 transmits and receives various types of information between one or more devices (hereinafter, also referred to as "second external devices") outside the medical support device 24 and the processor 82.

As one of the second external devices, the control device 22 is connected to the external I/F 80. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic video image 39 (see FIG. 1) from the processor 72 of the control device 22 via the external I/Fs 70 and 80 and performs various types of image processing on the acquired endoscopic video image 39.

As one of the second external devices, the display device 18 is connected to the external I/F 80. The processor 82 controls the display device 18 via the external I/F 80 such that various types of information (for example, the endoscopic video image 39 on which various types of image processing have been performed) are displayed on the display device 18.

The processing device 2 is connected to the external I/F 80 via the network 3 as one of the second external devices. The processor 82 exchanges various kinds of information with the processing device 2 via the external I/F 80. For example, the external I/F 80 transmits the endoscopic video image 39 acquired from the camera 52 to the processing device 2 by the processor 82. The processing device 2 receives the endoscopic video image 39 transmitted from the external I/F 80, executes processing using at least one frame 40 included in the received endoscopic video image 39, and transmits a processing result to the medical support device 24. The external I/F 80 receives the processing result transmitted from the processing device 2. The processor 82 acquires the processing result received by the external I/F 80.

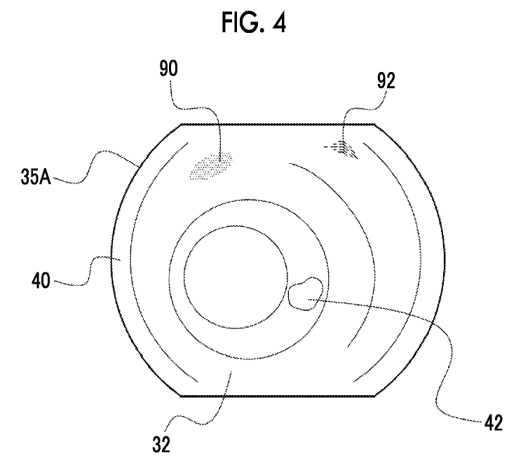

Incidentally, as shown in FIG. 4 as an example, the frame 40 displayed in the first display region 35A may show dirt 90 attached to the optical system 55 (for example, the illumination lens 55A and/or 55B) or may show a failure location 92 that is a location where the optical system 55 (for example, the illumination lens 55A and/or 55B) has failed. In a case where the dirt 90 or the failure location 92 is shown in the frame 40, the medical discrimination and/or treatment by the doctor 12 is hindered.

The dirt 90 attached to the optical system 55 can be wiped off by the in-hospital staff such as the doctor 12 or the nurse 14, but it is difficult for the in-hospital staff to deal with the failure of the optical system 55, and it is necessary to perform a specialized work such as replacement or repair of the optical system 55 by a specialized operator (hereinafter, also referred to as a "specialized operator") who handles the optical system.

Therefore, there is a demand for a unit that accurately diagnoses whether the failure of the optical system 55 is a failure that does not require the specialized operator to recover the optical system 55 or whether the failure of the optical system 55 is a failure that requires the specialized operator to recover the optical system 55 (in other words, whether what is shown in the frame 40 is the dirt 90 that can be easily dealt with by in-hospital staff or the failure location 92 that is difficult for in-hospital staff to deal with).

Therefore, in view of such circumstances, in the diagnostic system 1 according to the present embodiment, the optical system diagnosis is performed in the following manner. The optical system diagnosis refers to diagnosis for the optical system 55.

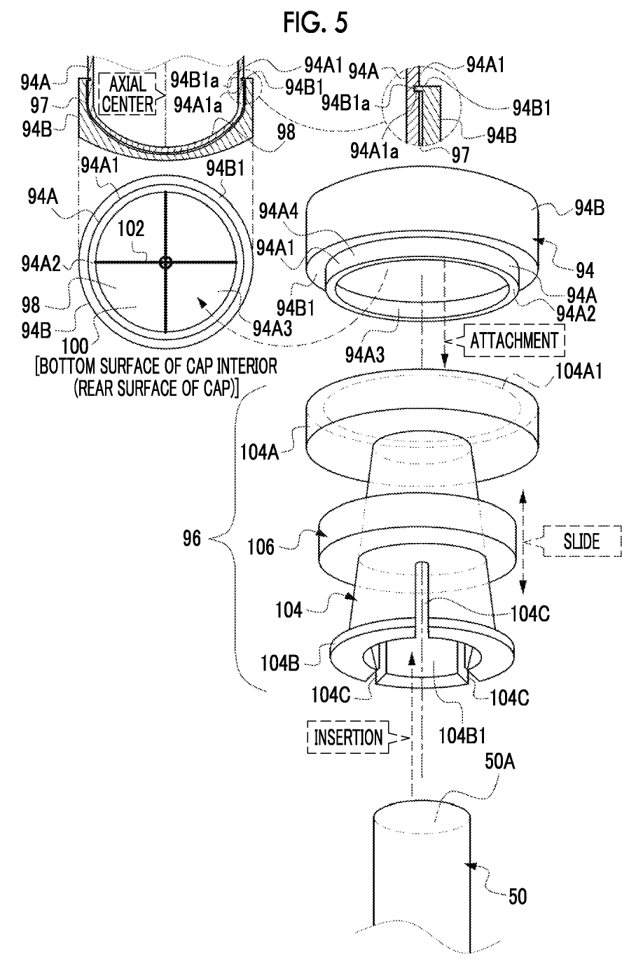

FIG. 5 is a conceptual diagram showing an example of configurations of a cap 94 and an attachment 96 used for the optical system diagnosis.

The cap 94 is mounted on the distal end portion 50 via the attachment 96. The cap 94 is formed in a two-layer structure including an inner layer 94A (in other words, an inner cap) and an outer layer 94B (in other words, an outer cap). Examples of materials of the inner layer 94A and the outer layer 94B include polyoxymethylene. Each of the inner layer 94A and the outer layer 94B is a bottomed cylindrical cap. The inner layer 94A is white. The outer layer 94B is black, and the outer layer 94B covers the inner layer 94A, so that external light is absorbed by the outer layer 94B. That is, since the color of the outer surface of the cap 94 is black, the external light is absorbed by the outer surface of the cap 94. In addition, a hollow region 97 is provided between the inner layer 94A and the outer layer 94B, and the hollow region 97 suppresses a change in the tone of the inner layer 94A due to the influence of the black color of the outer layer 94B. The inner layer 94A is an example of an "inner layer" according to the present disclosure, the outer layer 94B is an example of an "outer layer" according to the present disclosure, and the hollow region 97 is an example of a "hollow region" according to the present disclosure.

An annular flange 94B1 is formed on the outer layer 94B. The flange 94B1 has an annular protrusion 94B1a that protrudes in the inner diameter direction of the outer layer 94B (in other words, that protrudes perpendicularly to the axial center side of the outer layer 94B). An annular groove 94A1a is formed on an outer peripheral surface 94A1 of the inner layer 94A. In a case where the outer layer 94B is covered with the inner layer 94A such that the inner side of the outer layer 94B covers the outer side of the inner layer 94A, the protrusion 94B1a of the flange 94B1 is fitted into the groove 94A1a of the outer peripheral surface 94A1 of the inner layer 94A. As a result, the inner layer 94A is fixed to the outer layer 94B. It should be noted that, here, the form example is described in which the inner layer 94A is fixed to the outer layer 94B by fitting the protrusion 94B1a into the groove 94A1a, but this is merely an example, and the structure in which the outer layer 94B is covered with the inner layer 94A to be fixed may be another structure.

The inner layer 94A has an annular edge 94A2 having a diameter smaller than that of the flange 94B1. The edge 94A2 protrudes from the outer layer 94B to the outside along the axial center direction of the outer layer 94B. The inner layer 94A has an opening 94A3. The opening 94A3 is formed in a circular shape by the edge 94A2.

The inner layer 94A has an annular sleeve 94A4. The sleeve 94A4 is a portion that protrudes from the outer layer 94B to the outside along the axial center direction of the inner layer 94A, including the edge 94A2 of the inner layer 94A.

A rear surface 98 of the inner layer 94A (that is, the bottom of the inner layer 94A on the inner side) is formed in a curved shape that is recessed in a back side direction as viewed from the opening 94A3 side. Examples of the curved shape include an integral sphere shape. Here, a form example is described in which the rear surface 98 is formed in a curved shape, but this is merely an example, and the rear surface 98 may be formed in a planar shape.

The rear surface 98 has a first region 100 and a second region 102. The first region 100 is a region that reflects the light 30 (see FIG. 1). Here, an example of the light 30 reflected by the first region 100 is white light, but this is merely an example, and other types of light may be used. The second region 102 is a region having a lower reflectance than the first region 100. An area of the first region 100 is larger than an area of the second region 102. In addition, a spatial frequency of the second region 102 is higher than a spatial frequency of the first region 100. The second region 102 is a test chart. In the example shown in FIG. 5, a test chart having a cross shape and having a ring-shaped mark formed at the center is shown as the second region 102.

The rear surface 98 is a surface that diffuses the light 30 emitted from the endoscope 16. In the present embodiment, the rear surface 98 is subjected to a blasting treatment in order to diffuse the light 30.

The attachment 96 comprises a tubular structure 104 and a slide member 106. The tubular structure 104 has an upper side and a lower side that are open and penetrates from the upper side to the lower side. A material of the tubular structure 104 is a material (here, for example, a resin) that is extensible in a radial direction. A color of the tubular structure 104 is black. The tubular structure 104 is formed in a cylindrical shape, and an inner diameter of a body of the tubular structure 104 gradually increases from the upper side to the lower side.

An annular first flange 104A is formed at an upper end part of the tubular structure 104, and an annular second flange 104B having a smaller diameter than the first flange 104A is formed at a lower end part of the tubular structure 104.

A circular opening 104A1 for mounting the cap 94 on the tubular structure 104 is formed at a central portion of the first flange 104A. A diameter of the opening 104A1 is a diameter that allows the sleeve 94A4 to be inserted and allows an inner peripheral surface of the first flange 104A and an outer peripheral surface of the sleeve 94A4 inserted into the opening 104A1 to be closely attached to each other.

A circular opening 104B1 for inserting the distal end portion 50 of the endoscope 16 is formed at a central portion of the second flange 104B. The tubular structure 104 penetrates from the opening 104B1 to the opening 104A1.

A plurality of notches 104C are formed at a lower portion (that is, a second flange 104B side) of the tubular structure 104 at regular intervals around the central axis of the tubular structure 104 (that is, a circumferential direction of the tubular structure 104). The notches 104C are formed in a line shape along the axial direction of the tubular structure 104 from the second flange 104B side toward the first flange 104A side.

A slide member 106 is attached between the first flange 104A and the second flange 104B of the outer surface of the tubular structure 104 to be slidable along the axial direction of the tubular structure 104. The slide member 106 is formed of an elastic material such as a resin. The tubular structure 104 expands and contracts in the radial direction as the slide member 106 slides along the axial direction of the tubular structure 104.

The cap 94 is mounted on the tubular structure 104. That is, the cap 94 is mounted on the tubular structure 104 by inserting the sleeve 94A4 into the opening 104A1 against the pressure from the inner peripheral surface of the first flange 104A until the flange 94B1 comes into contact with the first flange 104A (that is, the sleeve 94A4 is press-fitted into the first flange 104A). Here, a form example is described in which the sleeve 94A4 is pressed against the inner peripheral surface of the first flange 104A to bring the outer peripheral surface of the sleeve 94A4 and the inner peripheral surface of the first flange 104A into close contact with each other, but this is merely an example. For example, one of a female screw and a male screw may be formed on the outer peripheral surface of the sleeve 94A4, the other of the female screw and the male screw may be formed on the inner peripheral surface of the first flange 104A, and the outer peripheral surface of the sleeve 94A4 may be fitted into the inner peripheral surface of the first flange 104A by using a screw structure.

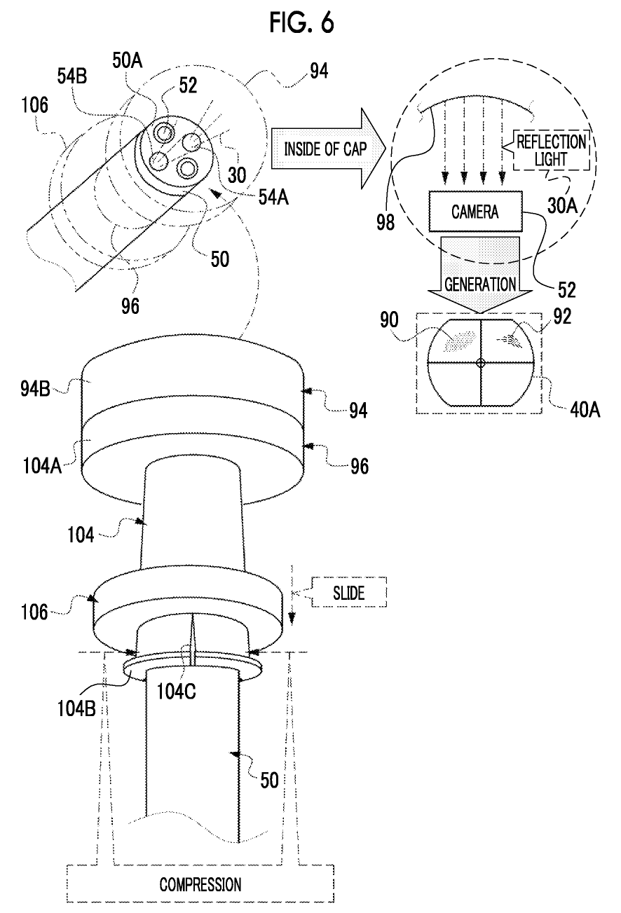

The distal end portion 50 of the endoscope 16 is inserted into the tubular structure 104 through the opening 104B1. Then, in a state in which the distal end portion 50 of the endoscope 16 is inserted into the tubular structure 104, the slide member 106 is slid along the axial direction of the tubular structure 104 to adjust the diameter of a portion of the attachment 96 that connects the cap 94 and the distal end portion 50. For example, by sliding the slide member 106 to the second flange 104B side along the axial direction of the tubular structure 104, the diameter of the portion where the cap 94 and the distal end portion 50 are connected to each other is reduced, and as a result, for example, as shown in FIG. 6, the distal end portion 50 of the endoscope 16 is sandwiched by the slide member 106 via the tubular structure 104 around the distal end portion 50 and is brought into close contact with the inner surface of the tubular structure 104.

In this way, the cap 94 is mounted on the distal end portion 50 via the attachment 96, so that the distal end surface 50A faces the rear surface 98 and an optically dense space is formed in the cap 94. In this situation, in a case where the distal end portion 50 is irradiated with the light 30 from the illumination lenses 55A and 55B, the light 30 is reflected by the rear surface 98 of the cap 94. The reflected light 30A obtained by reflecting the light 30 by the rear surface 98 is imaged by the camera 52. That is, the rear surface 98 is imaged by the camera 52. In this way, the diagnostic frame 40A is generated by imaging the rear surface 98 with the camera 52.

The diagnostic frame 40A is a frame used for diagnosing the optical system 55. In a case where the dirt 90 adheres to the optical system 55 (for example, the illumination lens 55A and/or 55B), the dirt 90 is shown in the diagnostic frame 40A, and in a case where the optical system 55 (for example, the illumination lens 55A and/or 55B) fails, the failure location 92 is shown in the diagnostic frame 40A. In this way, in a case where the dirt 90 and/or the failure location 92 is shown in the diagnostic frame 40A, the optical system 55 is diagnosed as being abnormal (that is, having a malfunction). In addition, in a case where neither the dirt 90 nor the failure location 92 is shown in the diagnostic frame 40A, the optical system 55 is diagnosed as being normal. In the present embodiment, the diagnostic frame 40A is an example of a "rear surface image" according to the present disclosure.

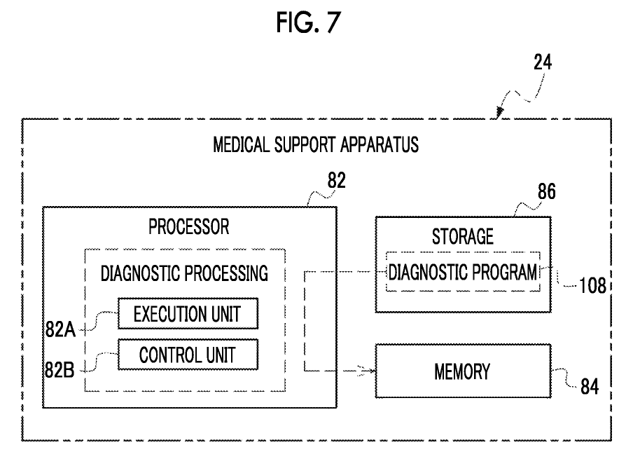

In order to realize such optical system diagnosis, in the present embodiment, as shown in FIG. 7 as an example, the processor 82 of the medical support device 24 performs diagnosis processing. FIG. 7 is a block diagram showing an example of functions of main units of the processor 82 included in the medical support device 24 and an example of information stored in the storage 86.

A diagnostic program 108 is stored in the storage 86. The diagnostic program 108 is an example of a "program" according to the present disclosure. The processor 82 reads out the diagnostic program 108 from the storage 86 and executes the readout diagnostic program 108 on the memory 84 to perform the diagnosis processing. The diagnosis processing is realized by the processor 82 operating as an execution unit 82A and a controller 82B in accordance with the diagnostic program 108 executed on the memory 84.

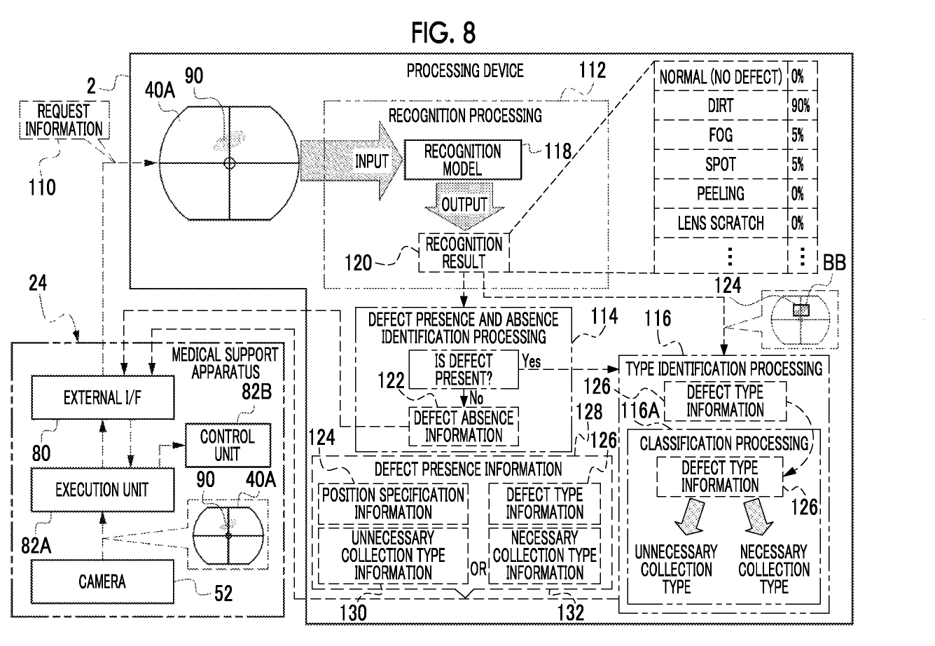

FIG. 8 is a conceptual diagram showing an example of the processing contents performed by the medical support device 24 and the processing device 2 in a state in which the cap 94 is mounted on the distal end portion 50 of the endoscope 16 via the attachment 96. Here, the state in which the cap 94 is mounted on the distal end portion 50 via the attachment 96 refers to a state in which the distal end surface 50A faces the rear surface 98 and a light-tight space is formed within the cap 94 (that is a state in which the distal end portion 50 is light-tightly closed by the cap 94 and the attachment 96).

As shown in FIG. 8, the execution unit 82A acquires the diagnostic frame 40A generated by being captured by the camera 52 at an imaging frame rate (for example, several tens of frames/second) in units of one frame in time series from the camera 52. Here, the diagnostic frame 40A included in the video is shown as an example, but this is merely an example, and the diagnostic frame 40A generated as a still image may be used.

The execution unit 82A indirectly executes the optical system diagnosis by using the processing device 2. That is, the execution unit 82A requests the processing device 2 to execute the optical system diagnosis and receives the result obtained by executing the optical system diagnosis by the processing device 2 from the processing device 2.

In a case in which the execution unit 82A acquires the diagnostic frame 40A from the camera 52, the execution unit 82A generates request information 110 and transmits the request information 110 to the processing device 2 via the external I/F 80. The request information 110 is information for requesting the processing device 2 to execute the optical system diagnosis. The request information 110 includes the diagnostic frame 40A acquired by the execution unit 82A.

The processing device 2 receives the request information 110 transmitted from the execution unit 82A. In a case in which the processing device 2 receives the request information 110, the processing device 2 executes the diagnosis of the optical system 55 by performing the image analysis using AI on the diagnostic frame 40A included in the request information 110. The processing device 2 executes recognition processing 112, defect presence and absence specification processing 114, and type identification processing 116 as the image analysis using AI. In the present embodiment, the diagnosis of the optical system 55 is realized by executing the recognition processing 112, the defect presence and absence specification processing 114, and the type identification processing 116 by the processing device 2 in response to the request from the execution unit 82A. In the present embodiment, the recognition processing 112, the defect presence and absence specification processing 114, and the type identification processing 116 are examples of "image analysis using AI" according to the present disclosure. In addition, in the present embodiment, the defect presence and absence specification processing 114 is an example of "malfunction presence/absence specification processing" according to the present disclosure, and the type identification processing 116 is an example of "type identification processing" and "position specification processing" according to the present disclosure.

The processing device 2 includes a recognition model 118. In the processing device 2, processing using the recognition model 118 is performed as the recognition processing 112. The processing device 2 performs the recognition processing 112 on the diagnostic frame 40A included in the request information 110 to recognize the type of malfunction of the optical system 55 including whether or not the optical system 55 is normal, and generates a recognition result 120. The type of malfunction of the optical system 55 including whether or not the optical system 55 is normal is recognized by the recognition model 118 with a confidence degree (for example, a probability).

The recognition model 118 is a trained model for object recognition in a bounding box method using AI. The recognition model 118 has been optimized by training a neural network through machine learning using first training data. The first training data is a data set including a plurality of data (that is, data for a plurality of frames) in which the first example data and the first correct answer data are associated with each other.

The first example data is an image assuming the diagnostic frame 40A. A first example of the image assuming the diagnostic frame 40A is an image obtained by imaging the rear surface 98 with a camera (that is, a camera having the same specifications as the camera 52) in a state in which the cap 94 is mounted on a distal end portion (that is, a portion corresponding to the distal end portion 50) of an endoscope having the same specifications as the endoscope 16 via the attachment 96. A second example of the image assuming the diagnostic frame 40A is an image that is virtually created (for example, an image generated by generative AI).

The first correct answer data is correct answer data (that is, an annotation) for the first example data. Here, examples of the first correct answer data include an annotation indicating whether or not an image used as the first example data shows a defect of the optical system 55 (for example, a defect corresponding to the dirt 90 and/or the failure location 92) and an annotation capable of specifying the type of the defect of the optical system 55 and the presence position of the defect of the optical system 55.

Examples of the type of the defect of the optical system 55 include the dirt 90, fogging (for example, fogging on a lens included in the optical system 55), a stain (for example, a stain on a lens included in the optical system 55), peeling (for example, peeling of a portion coating a surface of a lens included in the optical system 55, peeling of a cemented lens included in the optical system 55, and/or peeling between a lens included in the optical system 55 and a peripheral member), and a lens scratch (for example, a scratch on a lens included in the optical system 55).

The type of the defect of the optical system 55 is roughly classified into an unnecessary recovery type and a required recovery type. The unnecessary recovery type is a type of a defect that does not require the recovery of the optical system 55. The required recovery type is a type of a defect that requires the recovery of the optical system 55. An example of the unnecessary recovery type is the dirt 90. An example of the required recovery type is a defect other than the dirt 90, that is, a failure. Examples of the failure include fogging, a stain, peeling, and a lens scratch. Here, the unnecessary recovery type is an example of a "first type" according to the present disclosure, and the required recovery type is an example of a "second type" according to the present disclosure.

The processing device 2 acquires the diagnostic frame 40A from the request information 110 and inputs the acquired diagnostic frame 40A to the recognition model 118. As a result, the recognition model 118 recognizes the type of the defect of the optical system 55, including whether or not the optical system 55 is normal, for the input diagnostic frame 40A, and generates a recognition result 120. The recognition result 120 is an example of "information on which a diagnosis result is based" according to the present disclosure.

The defect presence and absence specification processing 114 is a process of specifying the presence or absence of the defect of the optical system 55 based on the recognition result 120 generated by the recognition model 118. In a case where it is determined that there is no defect in the optical system 55 by the defect presence and absence specification processing 114, the processing device 2 generates defect absence information 122 indicating that there is no defect in the optical system 55 and transmits the defect absence information 122 to the medical support device 24. The defect absence information 122 includes the diagnostic frame 40A input to the recognition model 118 in order to obtain the recognition result 120 used for determining that there is no defect in the optical system 55. The defect absence information 122 is received by the external I/F 80 of the medical support device 24. The defect absence information 122 received by the external I/F 80 is acquired by the execution unit 82A. Although details will be described below, in a case where the defect absence information 122 is acquired by the execution unit 82A, the controller 82B executes a process based on the defect absence information 122. In the present embodiment, the defect absence information 122 is an example of a "diagnosis result" according to the present disclosure.

In a case where it is determined that there is a defect in the optical system 55 by the defect presence and absence specification processing 114, the processing device 2 executes a type identification processing 116. The type identification processing 116 is a process of specifying the type of the defect of the optical system 55 based on the recognition result 120 generated by the recognition model 118 and specifying a position where the defect of the optical system 55 occurs on the diagnostic frame 40A. The type identification processing 116 specifies the position where the defect of the optical system 55 occurs on the diagnostic frame 40A by acquiring position specification information 124 capable of specifying the position where the defect of the optical system 55 occurs on the diagnostic frame 40A from the recognition result 120. The position specification information 124 includes a bounding box BB capable of specifying the position where the defect of the optical system 55 occurs on the diagnostic frame 40A. In the type identification processing 116, the type of the defect of the optical system 55 is specified based on the recognition result 120, and defect type information 126 indicating the type of the defect of the optical system 55 (for example, the dirt 90, the fog, the stain, the peeling, the lens scratch, or the like) is generated.

The type identification processing 116 includes a splitting processing 116A. The splitting processing 116A is a process of splitting the type of the defect of the optical system 55 into an unnecessary recovery type and a required recovery type. The splitting of the unnecessary recovery type and the required recovery type is performed based on the defect type information 126. For example, in a case where the dirt 90 is specified as the type of the defect of the optical system 55 by the defect type information 126, the type of the defect of the optical system 55 is classified into the unnecessary recovery type, and in a case where a type other than the dirt 90 is specified as the type of the defect of the optical system 55 by the defect type information 126, the type of the defect of the optical system 55 is classified into the required recovery type.

In a case where the type identification processing 116 is executed in this way to specify the type of the defect of the optical system 55, specify the position where the defect of the optical system 55 occurs on the diagnostic frame 40A, and split the type of the defect of the optical system 55 into the unnecessary recovery type or the required recovery type, the processing device 2 generates defect presence information 128 as a processing result of the type identification processing 116 and transmits the defect presence information 128 to the medical support device 24.

The defect presence information 128 includes the position specification information 124 and the defect type information 126. In addition, the defect presence information 128 includes unnecessary collection type information 130 or necessary collection type information 132. The unnecessary collection type information 130 is information indicating that the type of the defect of the optical system 55 is classified into the unnecessary recovery type by the splitting processing 116A, and the necessary collection type information 132 is information indicating that the type of the defect of the optical system 55 is classified into the required recovery type by the splitting processing 116A.

The defect presence information 128 transmitted by the processing device 2 is received by the external I/F 80 of the medical support device 24. The execution unit 82A acquires the defect presence information 128 received by the external I/F 80. Although details will be described below, in a case where the execution unit 82A acquires the defect presence information 128, the controller 82B executes processing based on the defect presence information 128. In the present embodiment, the defect presence information 128 is an example of a "diagnosis result" according to the present disclosure.

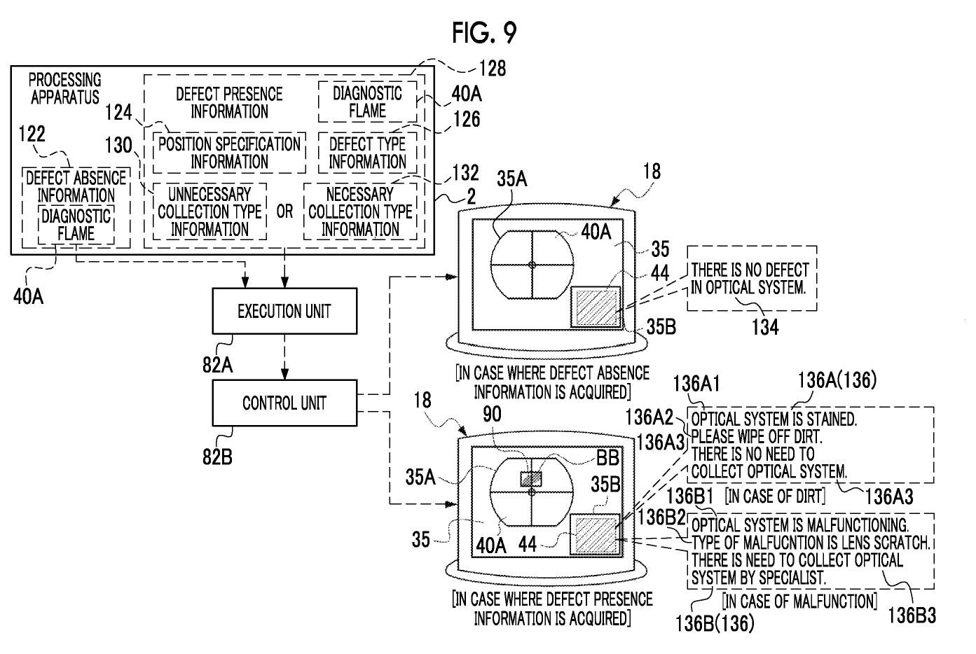

FIG. 9 is a conceptual diagram showing an example of display control performed by the controller 82B on the display device 18 in a case where the defect absence information 122 or the defect presence information 128 is acquired by the execution unit 82A. As shown in FIG. 9, in a case where the execution unit 82A acquires the defect absence information 122, the controller 82B outputs the defect absence information 122 (that is, the defect absence information 122 obtained by executing the recognition processing 112 and the defect presence and absence specification processing 114) obtained by executing the diagnosis of the optical system 55 by the processing device 2, as visualized information.

That is, in a case where the execution unit 82A acquires the defect absence information 122, the controller 82B displays the diagnostic frame 40A included in the defect absence information 122 in the first display region 35A, and displays the defect absence message 134 as one of the auxiliary information 44 in the second display region 35B. The defect absence message 134 is a message indicating that there is no defect in the optical system 55. The diagnostic frame 40A displayed in the first display region 35A and the defect absence message 134 displayed in the second display region 35B are examples of the defect absence information 122 as visualized information.

Here, although a form example is described in which the defect absence message 134 is displayed in the second display region 35B, this is merely an example, and a mark and/or a code indicating that there is no defect in the optical system 55 may be displayed in the second display region 35B. In addition, a voice indicating that there is no defect in the optical system 55 may be output from a speaker (not shown). In addition, the defect absence information 122 and/or information (for example, the defect absence message 134) generated based on the defect absence information 122 may be stored in a storage region (for example, the storage 86).

On the other hand, in a case where the execution unit 82A acquires the defect presence information 128, the controller 82B outputs the defect presence information 128 (that is, the defect presence information 128 obtained by executing the recognition processing 112, the defect presence and absence specification processing 114, and the type identification processing 116) obtained by executing the diagnosis of the optical system 55 by the processing device 2, as visualized information.

That is, in a case where the execution unit 82A acquires the defect presence information 128, the controller 82B displays the diagnostic frame 40A included in the defect presence information 128 in the first display region 35A, and displays the defect presence message 136 as one of the auxiliary information 44 in the second display region 35B. The controller 82B displays a bounding box BB in a superimposed manner on the diagnostic frame 40A displayed in the first display region 35A based on the position specification information 124 included in the defect presence information 128.

In the example shown in FIG. 9, a form example is shown in which the bounding box BB is displayed at a portion where the dirt 90 is shown on the diagnostic frame 40A, but in a case where the failure of the optical system 55 is specified by the defect type information 126, the controller 82B displays the bounding box BB in a superimposed manner at the failure location 92 (see FIG. 4) on the diagnostic frame 40A. In this case, the controller 82B makes a display aspect of the bounding box BB displayed at the portion where the dirt 90 is shown and a display aspect of the bounding box BB displayed at the failure location 92 different from each other such that the bounding box BB displayed at the portion where the dirt 90 is shown and the bounding box BB displayed at the failure location 92 can be visually distinguished from each other. For example, the controller 82B visually differentiates the bounding box BB displayed at the portion where the dirt 90 is shown and the bounding box BB displayed at the failure location 92 by changing a line thickness, a line type, a brightness, and/or a color of the bounding box BB. In addition, a case where one or more dirt 90 and one or more failure locations 92 are shown in the diagnostic frame 40A is also considered. In this case as well, the display aspect of the bounding box BB displayed at the portion where the dirt 90 is shown and the display aspect of the bounding box BB displayed at the failure location 92 may be made different from each other in the same manner.

The defect presence message 136 is a message indicating that the optical system 55 has a defect. The diagnostic frame 40A displayed in the first display region 35A and the defect presence message 136 displayed in the second display region 35B are examples of visualized information of the defect presence information 128.

The defect presence message 136 is classified into a dirt presence message 136A and a failure presence message 136B. In a case where the defect presence information 128 is acquired by the execution unit 82A, any one of the dirt presence message 136A or the failure presence message 136B is displayed in the second display region 35B. In a case where the information indicated by the defect type information 126 included in the defect presence information 128 is information indicating the dirt 90, the controller 82B displays the dirt presence message 136A in the second display region 35B. In a case where the information indicated by the defect type information 126 included in the defect presence information 128 is information indicating a failure (for example, fog, a stain, peeling, a lens scratch, or the like), the controller 82B displays the failure presence message 136B in the second display region 35B.

The dirt presence message 136A includes a first message 136A1 indicating that the dirt 90 is attached to the optical system 55, a second message 136A2 recommending wiping off the dirt 90, and a third message 136A3 indicating that the optical system 55 does not need to be recovered. The first message 136A1 and the second message 136A2 are information generated by the controller 82B based on the defect type information 126 included in the defect presence information 128. That is, the first message 136A1 and the second message 136A2 can be said to be visualized information of the defect type information 126. The third message 136A3 is information generated by the controller 82B based on the unnecessary collection type information 130 included in the defect presence information 128. That is, the third message 136A3 can be said to be visualized information of the unnecessary collection type information 130.

The failure presence message 136B includes a fourth message 136B1 indicating that the optical system 55 has failed, a fifth message 136B2 for specifying a type of the failure of the optical system 55, and a sixth message 136B3 indicating that the optical system 55 needs to be recovered by a professional operator. The fourth message 136B1 and the fifth message 136B2 are information generated by the controller 82B based on the defect type information 126 included in the defect presence information 128. That is, the fourth message 136B1 and the fifth message 136B2 can be said to be visualized information of the defect type information 126. The sixth message 136B3 is information generated by the controller 82B based on the necessary collection type information 132 included in the defect presence information 128. That is, the sixth message 136B3 can be said to be visualized information of the necessary collection type information 132.

It should be noted that, here, the form example is described in which the defect presence message 136 is displayed in the second display region 35B, but this is merely an example, and a mark and/or a code indicating that the optical system 55 has a defect may be displayed in the second display region 35B. In addition, a voice indicating the first message 136A1, the second message 136A2, the third message 136A3, the fourth message 136B1, the fifth message 136B2, and/or the sixth message 136B3 may be output from a speaker (not shown). In addition, the defect presence information 128 and/or the information generated based on the defect presence information 128 (for example, the first message 136A1, the second message 136A2, the third message 136A3, the fourth message 136B1, the fifth message 136B2, and/or the sixth message 136B3) may be stored in a storage region (for example, the storage 86).

The content (that is, the diagnosis result of the optical system 55) displayed in the first display region 35A and the second display region 35B by the controller 82B is referred to and evaluated by a professional operator of the optical system (hereinafter, also referred to as a "professional operator"). For example, the professional operator evaluates the validity of the content (that is, the diagnosis result of the optical system 55) displayed in the first display region 35A and the second display region 35B. The evaluation result by the professional operator is received by the reception device 64 or the like. The execution unit 82A causes the processing device 2 to execute retraining of the recognition model 118 based on the evaluation result received by the reception device 64 or the like. As a result, the recognition model 118 is strengthened.

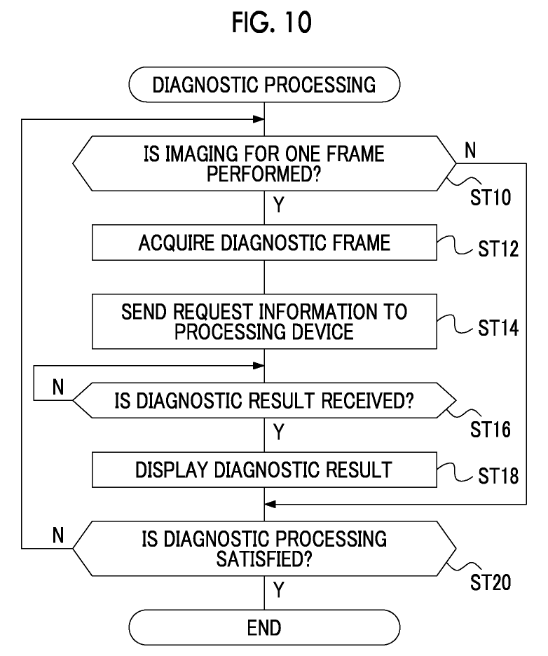

Next, an action of a part of the diagnostic system 1 according to the present disclosure will be described with reference to FIG. 10. A flow of the diagnosis processing shown in FIG. 10 is an example of a "diagnosis method" according to the present disclosure.

In the diagnosis processing shown in FIG. 10, first, in step ST10, the execution unit 82A determines whether or not imaging for one frame in which the rear surface 98 is used as a subject is performed by the camera 52 in a state in which the cap 94 is mounted on the distal end portion 50 of the endoscope 16 via the attachment 96. In step ST10, in a case in which the imaging for one frame in which the rear surface 98 is used as a subject is not performed by the camera 52 in a state in which the cap 94 is mounted on the distal end portion 50 of the endoscope 16 via the attachment 96, a negative determination is made, and the diagnosis processing proceeds to step ST20. In step ST10, in a case in which the imaging for one frame in which the rear surface 98 is used as a subject is performed by the camera 52 in a state in which the cap 94 is mounted on the distal end portion 50 of the endoscope 16 via the attachment 96, a positive determination is made, and the diagnosis processing proceeds to step ST12.

In step ST12, the execution unit 82A acquires the diagnostic frame 40A obtained by imaging the rear surface 98 via the camera 52. After the processing of step ST12 is executed, the diagnosis processing proceeds to step ST14.

In step ST14, the execution unit 82A transmits the request information 110 including the diagnostic frame 40A acquired in step ST12 to the processing device 2 via the external I/F 80. After the processing of step ST14 is executed, the diagnosis processing proceeds to step ST16.

In a case in which the request information 110 is transmitted to the processing device 2 by executing the processing of step ST14, the processing device 2 performs the recognition processing 112 on the diagnostic frame 40A included in the request information 110, and performs the defect presence and absence specification processing 114 using the recognition result 120. In a case in which it is determined that there is no malfunction in the optical system 55 by performing the defect presence and absence specification processing 114, the defect absence information 122 is transmitted to the medical support device 24 as the diagnosis result. In addition, in a case in which it is determined that there is a malfunction in the optical system 55 by performing the defect presence and absence specification processing 114, the type identification processing 116 using the recognition result 120 is performed. The defect presence information 128 is generated by performing the type identification processing 116 and is transmitted to the medical support device 24 as the diagnosis result.

Therefore, in step ST16, the execution unit 82A determines whether or not the diagnosis result (that is, the defect absence information 122 or the defect presence information 128) transmitted from the processing device 2 is received by the external I/F 80. In step ST16, in a case in which the diagnosis result transmitted from the processing device 2 is not received by the external I/F 80, a negative determination is made, and the determination in step ST16 is performed again. In step ST16, in a case in which the diagnosis result transmitted from the processing device 2 is received by the external I/F 80, an affirmative determination is made, and the diagnosis processing proceeds to step ST18. In a case in which the diagnosis result transmitted from the processing device 2 is received by the external I/F 80, the diagnosis result received by the external I/F 80 is acquired by the execution unit 82A.

In step ST18, the controller 82B displays the diagnosis result (that is, the defect absence information 122 or the defect presence information 128) acquired by the execution unit 82A on the screen 35 as the visualized information (see FIG. 9). After the processing in step ST18 is executed, the diagnosis processing proceeds to step ST20.

In step ST20, the controller 82B determines whether or not a condition for ending the diagnosis processing is satisfied. Examples of the condition for ending the diagnosis processing include a condition in which an instruction to end the diagnosis processing is given to the diagnostic system 1 (for example, a condition in which the instruction to end the diagnosis processing is received by the reception device 64).

In step ST20, in a case in which the condition for ending the diagnosis processing is not satisfied, a negative determination is made, and the diagnosis processing proceeds to step ST10. In step ST20, in a case in which the condition for ending the diagnosis processing is satisfied, an affirmative determination is made, and the diagnosis processing ends.

As described above, in the diagnostic system 1, the diagnosis of the optical system 55 is executed by performing the image analysis using the AI on the diagnostic frame 40A obtained by imaging the rear surface 98 with the camera 52 in a state in which the cap 94 is mounted on the distal end portion 50 of the endoscope 16 via the attachment 96. Then, the diagnosis result obtained by executing the diagnosis is displayed on the screen 35. The diagnosis result displayed on the screen 35 is the information in which the defect absence information 122 is visualized or the information in which the defect presence information 128 is visualized. Therefore, the doctor 12, the professional operator, and the like can accurately diagnose the optical system 55 by visually recognizing the information in which the defect absence information 122 is visualized or the information in which the defect presence information 128 is visualized through the screen 35.

In addition, in the diagnostic system 1, the processing device 2 executes the defect presence and absence specification processing 114. The defect presence and absence specification processing 114 is a process of specifying the presence or absence of the defect of the optical system 55 based on the recognition result 120 generated by the recognition model 118. In a case in which it is determined that the defect is present in the optical system 55 by performing the defect presence and absence specification processing 114, the defect presence information 128 is generated by the processing device 2 and transmitted to the medical support device 24. In a case in which it is determined that the defect is not present in the optical system 55 by performing the defect presence and absence specification processing 114, the defect absence information 122 is generated by the processing device 2 and transmitted to the medical support device 24. The defect absence information 122 or the defect presence information 128 is displayed on the screen 35 as the visualized information. As a result, the doctor 12, the professional operator, and the like can understand the presence or absence of the defect in the optical system 55.

In addition, in the diagnostic system 1, the processing device 2 executes the type identification processing 116. The type identification processing 116 includes processing of specifying the type of the defect in the optical system 55 based on the recognition result 120 generated by the recognition model 118. The type of the defect in the optical system 55 is classified into an unnecessary recovery type and a required recovery type. The unnecessary recovery type is a type of a defect that does not require the recovery of the optical system 55. The required recovery type is a type of a defect that requires the recovery of the optical system 55. The type identification processing 116 includes a splitting processing 116A, and the type of the defect occurring in the optical system 55 is split into the unnecessary recovery type and the required recovery type by executing the splitting processing 116A, and the result of the splitting is displayed on the screen 35. Therefore, in a case in which the defect occurs in the optical system 55, the doctor 12, the professional operator, and the like can understand whether the defect that does not require the recovery of the optical system 55 occurs or the defect that requires the recovery of the optical system 55 occurs.

Here, an example of the unnecessary recovery type is the dirt 90, and an example of the required recovery type is the failure. Then, whether the defect occurring in the optical system 55 is the dirt 90 or the failure is displayed on the screen 35. Therefore, in a case in which the defect occurs in the optical system 55, the doctor 12, the professional operator, and the like can understand whether the defect occurring in the optical system 55 is the dirt or the failure.

In addition, in the diagnostic system 1, the image analysis using the AI for the diagnostic frame 40A is realized by inputting the diagnostic frame 40A to the recognition model 118 to generate the recognition result 120. As a result, the type of the defect of the optical system 55 is specified quickly and accurately, including whether or not the optical system 55 is normal, as compared with a case in which the type of the defect of the optical system 55 is specified only based on the human intuition and the experience (for example, a case in which the human specifies the type of the defect of the optical system 55 including whether or not the optical system 55 is normal while visually checking the diagnostic frame 40A).

In addition, in the diagnostic system 1, the diagnostic result of the optical system 55 is referred to and evaluated by the professional operator through the screen 35. The evaluation result by the professional operator is received by the reception device 64 or the like. The execution unit 82A causes the processing device 2 to execute retraining of the recognition model 118 based on the evaluation result received by the reception device 64 or the like. As a result, the recognition model 118 is strengthened. In this way, the accuracy of the diagnosis of the optical system 55 can be improved.

In addition, in the diagnostic system 1, the type identification processing 116 is executed by the processing device 2, so that the processing device 2 acquires the position specification information 124 and transmits the position specification information 124 to the medical support device 24. The position specification information 124 is information for specifying a position at which the defect of the optical system 55 occurs on the diagnostic frame 40A, and is displayed in the first display region 35A as the visualized information (for example, the bounding box BB). As a result, the doctor 12, the professional operator, and the like can ascertain the position at which the defect of the optical system 55 occurs.

In addition, in the diagnostic system 1, the cap 94 is mounted on the distal end portion 50 via the attachment 96. The rear surface 98 of the cap 94 has the first region 100 and the second region 102 formed as the test chart. The second region 102 has a lower reflectance than the first region 100, has a smaller area than the first region 100, and has a higher spatial frequency than the first region 100. In the diagnostic system 1, the diagnosis of the optical system 55 is performed using the diagnostic frame 40A generated by imaging the rear surface 98 configured as described above with the camera 52. Therefore, it is possible to accurately specify the presence or absence of the defect of the optical system 55 and the type of the defect of the optical system 55. In particular, among the defects occurring on the surface of the lens included in the optical system 55, the dirt tends to be displayed on the screen 35 (here, as an example, the first display region 35A) in a light gray gradation close to white. Therefore, the determination accuracy of whether or not the defect is dirt is lower than a certain level only with the first region 100 (for example, the white region) having a higher reflectance than the second region 102. Therefore, in order to realize the determination accuracy equal to or higher than a certain level, in the present embodiment, the second region 102 having a lower reflectance than the first region 100 is also included in the test chart. As a result, it is possible to increase the determination accuracy of whether or not the defect is dirt to a certain level or higher.

In addition, the rear surface 98 of the cap 94 is formed in a curved shape. In addition, the rear surface 98 of the cap 94 is processed into a surface that diffuses the light 30. Therefore, it is possible to suppress the deterioration of the image quality of the diagnostic frame 40A due to the diffuse reflection of the light in the cap 94 or the like, which is not suitable for the diagnosis of the defect of the optical system 55.

In addition, the cap 94 is formed in a two-layer structure. In addition, the color of the outer surface of the cap 94 is black. Therefore, it is possible to suppress the incidence of the external light into the cap 94. In addition, the two-layer structure of the cap 94 is a two-layer structure including an inner layer 94A and an outer layer 94B, and a hollow region 97 is provided between the inner layer 94A and the outer layer 94B. As a result, it is possible to enhance the effect of suppressing the incidence of the external light into the cap 94.

In addition, the cap 94 is connected to the distal end portion 50 of the endoscope 16 via the attachment 96, and the diameter of a portion where the cap 94 and the distal end portion 50 are connected to each other by the attachment 96 is adjustable. Therefore, by adjusting the diameter of the portion where the cap 94 and the distal end portion 50 are connected to each other by the attachment 96, it is possible to facilitate the work of mounting the cap 94 on the distal end portion 50. In addition, by adjusting the diameter of the portion where the cap 94 and the distal end portion 50 are connected to each other by the attachment 96, it is possible to increase the degree of close attachment between the distal end portion 50 and the attachment 96, and thus it is possible to suppress the incidence of the external light into the cap 94 from the gap between the distal end portion 50 and the attachment 96.

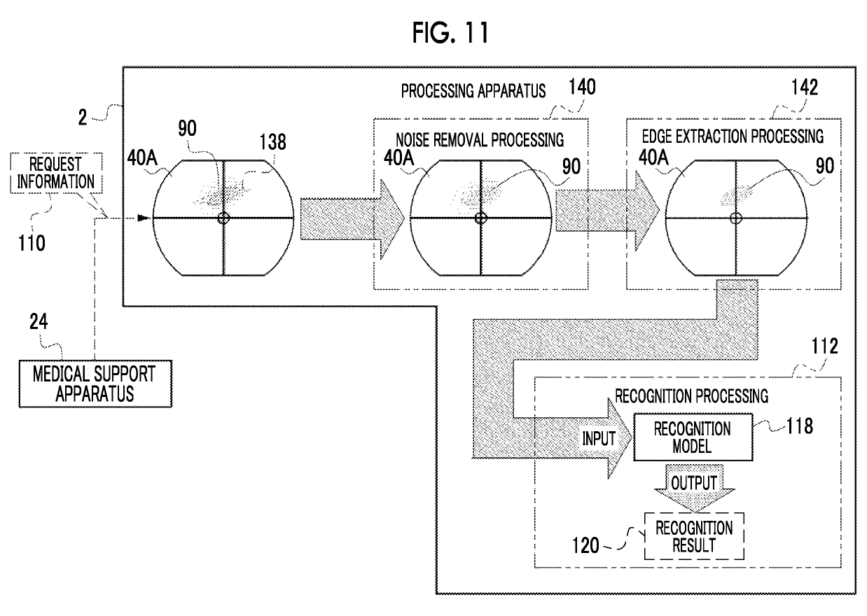

In the above-described embodiment, the form example has been described in which the processing device 2 inputs the diagnostic frame 40A included in the request information 110 transmitted from the medical support device 24 to the recognition model 118, but the present disclosure is not limited to this. For example, as shown in FIG. 11, the processing device 2 may perform noise removal processing 140 and edge extraction processing 142 on the diagnostic frame 40A in a stage before performing the recognition processing 112. In the example shown in FIG. 11, noise 138 is shown in the diagnostic frame 40A in addition to the dirt 90. The noise removal processing 140 is processing of removing the noise 138 from the diagnostic frame 40A. The edge extraction processing 142 is processing of extracting an edge of an image region shown in the diagnostic frame 40A from which the noise 138 is removed by performing the noise removal processing 140. The processing device 2 inputs the diagnostic frame 40A obtained by performing the edge extraction processing 142 to the recognition model 118. In this way, the accuracy of the recognition processing 112 is increased, and as a result, the accuracy of the diagnosis of the malfunction occurring in the optical system 55 is also increased.

Here, the form example has been described in which both the noise removal processing 140 and the edge extraction processing 142 are performed in the stage before the recognition processing 112, but this is merely an example, and the noise removal processing 140 or the edge extraction processing 142 may be performed in the stage before the recognition processing 112. The noise removal processing 140 is more effective when performed before the edge extraction processing 142 than when performed after the edge extraction processing 142.

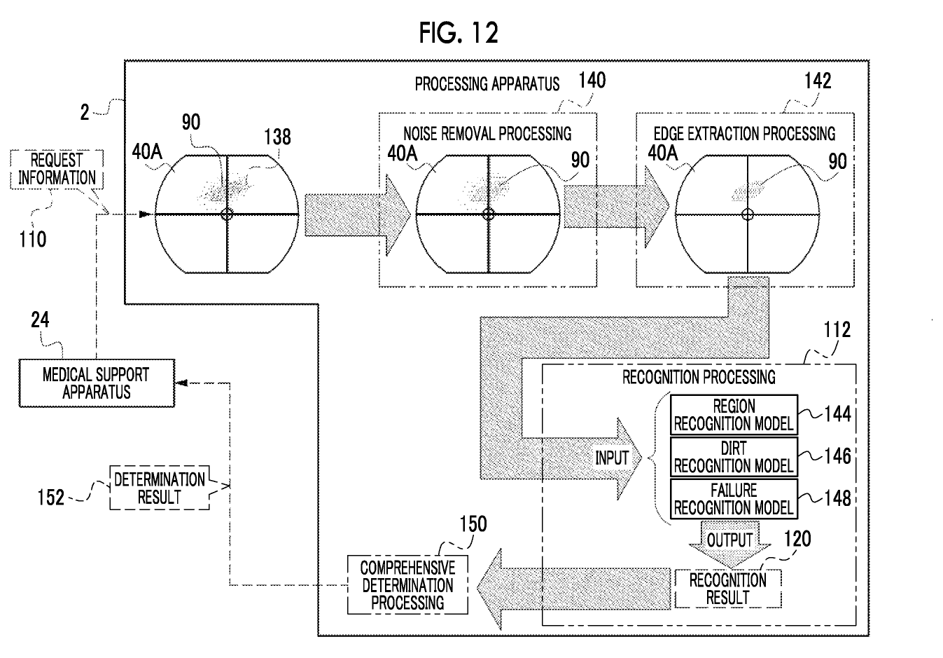

In the above-described embodiment, the form example has been described in which the recognition processing 112 uses the recognition model 118, but the present disclosure is not limited thereto. For example, as shown in FIG. 12, the recognition processing 112 may use a region recognition model 144, a dirt recognition model 146, and a failure recognition model 148 instead of the recognition model 118. In this case, the diagnostic frame 40A (in the example shown in FIG. 12, the diagnostic frame 40A from which the noise removal processing 140 and the edge extraction processing 142 are performed) is input to each of the region recognition model 144, the dirt recognition model 146, and the failure recognition model 148.

The region recognition model 144 is a trained model for object recognition in a bounding box method using AI. The region recognition model 144 has been optimized by training a neural network through machine learning using second training data. The second training data is a data set including a plurality of data items (that is, data corresponding to a plurality of frames) in which second example data and second correct answer data have been associated with each other.

The second example data is the same image as the first example data described in the above-described embodiment. The second correct answer data refers to correct answer data (that is, an annotation) for the second example data. Here, examples of the second correct answer data include an annotation capable of specifying an image region corresponding to the first region 100 shown in the image used as the second example data and an annotation capable of specifying an image region corresponding to the second region 102.

The processing device 2 acquires the diagnostic frame 40A from the request information 110 and inputs the acquired diagnostic frame 40A to the region recognition model 144. As a result, the region recognition model 144 recognizes the first region 100 and the second region 102 shown in the input diagnostic frame 40A.

The dirt recognition model 146 is a trained model for object recognition in a bounding box method using AI. The dirt recognition model 146 has been optimized by training a neural network through machine learning using third training data. The third training data is a dataset including a plurality of data (that is, data for a plurality of frames) in which third example data is associated with third ground truth data.

The third example data is the same image as the first example data described in the above-described embodiment. The third correct answer data is correct answer data (that is, an annotation) for the third example data. Here, examples of the third correct answer data include an annotation capable of specifying an image region corresponding to the dirt 90 shown in the image used as the third example data.

The dirt recognition model 146 configured in this way is AI (for example, AI that specifies a position where the dirt 90 is present on the diagnostic frame 40A) that specifies the dirt 90 based on the diagnostic frame 40A. The processing device 2 acquires the diagnostic frame 40A from the request information 110 and inputs the acquired diagnostic frame 40A to the dirt recognition model 146. Accordingly, the dirt recognition model 146 recognizes the dirt 90 shown in the input diagnostic frame 40A.

The failure recognition model 148 is a trained model for object recognition in a bounding box method using AI. The failure recognition model 148 has been optimized by training a neural network through machine learning using fourth training data. The fourth training data is a data set including a plurality of data items (that is, data corresponding to a plurality of frames) in which fourth example data and fourth correct answer data have been associated with each other.

The fourth example data is the same image as the first example data described in the above-described embodiment. The fourth correct answer data is correct answer data (that is, an annotation) for the fourth example data. Here, examples of the fourth correct answer data include an annotation capable of specifying an image region corresponding to each of various types of failures (for example, fog, stain, peeling, lens scratch, and the like) shown in the image used as the fourth example data.

The failure recognition model 148 configured as described above is AI (for example, AI that specifies a position where the failure location 92 (see FIG. 4) is present on the diagnostic frame 40A and a type of the failure) that specifies the failure of the optical system 55 based on the diagnostic frame 40A. The processing device 2 acquires the diagnostic frame 40A from the request information 110 and inputs the acquired diagnostic frame 40A to the failure recognition model 148. Accordingly, the failure recognition model 148 recognizes the failure shown in the input diagnostic frame 40A by type (for example, by type such as fog, stain, peeling, and lens scratch).

The recognition processing 112 compiles the recognition result by the region recognition model 144, the recognition result by the dirt recognition model 146, and the recognition result by the failure recognition model 148 as the recognition result 120 described in the above-described embodiment. The processing device 2 performs comprehensive determination processing 150 using the recognition result 120. The comprehensive determination processing 150 is processing of performing comprehensive determination on the recognition result 120. Examples of the comprehensive determination processing 150 include processing corresponding to the defect presence and absence specification processing 114 and the type identification processing 116 described in the above-described embodiment. The comprehensive determination processing 150 generates a determination result 152, which is information including the defect absence information 122 and the defect presence information 128, based on the recognition result 120. The processing device 2 transmits the determination result 152 to the medical support device 24. The medical support device 24 performs display control on the display device 18 in the same manner as in the above-described embodiment using the determination result 152.

In the example shown in FIG. 12, the dirt recognition model 146 is an example of a "first type-specific AI" according to the present disclosure, and the failure recognition model 148 is an example of a "second type-specific AI" according to the present disclosure.

As described above, since the dirt recognition model 146 is the AI specialized in recognizing the dirt 90 and the failure recognition model 148 is the AI specialized in recognizing the failure location 92, the dirt 90 and the failure of the optical system 55 can be accurately specified by using the dirt recognition model 146 and the failure recognition model 148 in combination.

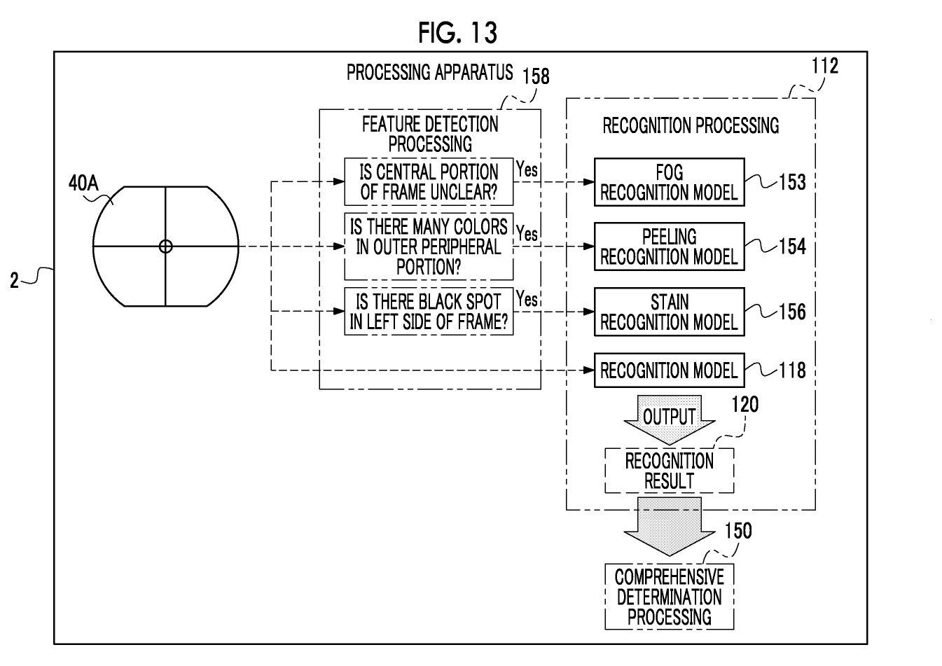

The failure recognition model 148 may be a recognition model that is patented for each type of failure. For example, as shown in FIG. 13, the recognition processing 112 may use, in addition to the recognition model 118, a fog recognition model 153 that is optimized by performing machine learning specialized in recognizing fog, a peeling recognition model 154 that is optimized by performing machine learning specialized in recognizing peeling, and a stain recognition model 156 that is optimized by performing machine learning specialized in recognizing stain.

It is known that, in a case where fog occurs in the optical system 55, a front view central portion of the diagnostic frame 40A displayed in the first display region 35A becomes unclear. In addition, it is known that, in a case where peeling occurs in the optical system 55, a chromatic region is reflected in the outer peripheral portion of the diagnostic frame 40A displayed in the first display region 35A at a certain level or higher. Further, it is known that, in a case where a stain occurs in the optical system 55, a black spot is reflected on a left side of the diagnostic frame 40A displayed in the first display region 35A in a front view.

Therefore, the processing device 2 performs feature detection processing 158 of detecting a feature reflected in the diagnostic frame 40A in a pre-stage of the recognition processing 112. In the feature detection processing 158, it is determined whether or not the front view central portion of the diagnostic frame 40A is unclear. Here, in a case where it is determined that the front view central portion of the diagnostic frame 40A is unclear, in the recognition processing 112, processing using the fog recognition model 153 is performed on the diagnostic frame 40A. In this case, the diagnostic frame 40A is input to the fog recognition model 153. Accordingly, the fog recognition model 153 determines the presence or absence of fog in the optical system 55, and specifies a position where fog is reflected on the diagnostic frame 40A in a case where it is determined that there is fog in the optical system 55.

In addition, in the feature detection processing 158, it is determined whether or not a chromatic region is reflected in the outer peripheral portion of the diagnostic frame 40A at a certain level or higher. Here, in a case where it is determined that a chromatic region is reflected in the outer peripheral portion of the diagnostic frame 40A at a certain level or higher, in the recognition processing 112, processing using the peeling recognition model 154 is performed on the diagnostic frame 40A. In this case, the diagnostic frame 40A is input to the peeling recognition model 154. Accordingly, the peeling recognition model 154 determines the presence or absence of peeling of the optical system 55, and specifies a position where peeling is reflected on the diagnostic frame 40A in a case where it is determined that there is peeling in the optical system 55.

In addition, in the feature detection processing 158, it is determined whether or not a black spot is reflected on the front view left side of the diagnostic frame 40A. Here, in a case where it is determined that a large number of black spots are reflected on the front view left side of the diagnostic frame 40A, in the recognition processing 112, processing using the stain recognition model 156 is performed on the diagnostic frame 40A. In this case, the diagnostic frame 40A is input to the stain recognition model 156. Accordingly, the stain recognition model 156 determines the presence or absence of a stain in the optical system 55, and specifies a position where a stain is reflected on the diagnostic frame 40A in a case where it is determined that there is a stain in the optical system 55.

In the recognition processing 112, the recognition result obtained by the recognition model 118 is reflected with the recognition result obtained by the fog recognition model 153, the recognition result obtained by the peeling recognition model 154, and the recognition result obtained by the stain recognition model 156 in the same manner as in the above-described embodiment. For example, the recognition result 120 is adjusted by assigning each of the recognition result obtained by the fog recognition model 153, the recognition result obtained by the peeling recognition model 154, and the recognition result obtained by the stain recognition model 156 as a weight to the recognition result 120. The recognition result 120 obtained in this way is used in the comprehensive determination processing 150. Then, the comprehensive determination processing 150 is performed to generate the determination result 152, and the determination result 152 is transmitted to the medical support device 24 (see FIG. 12).

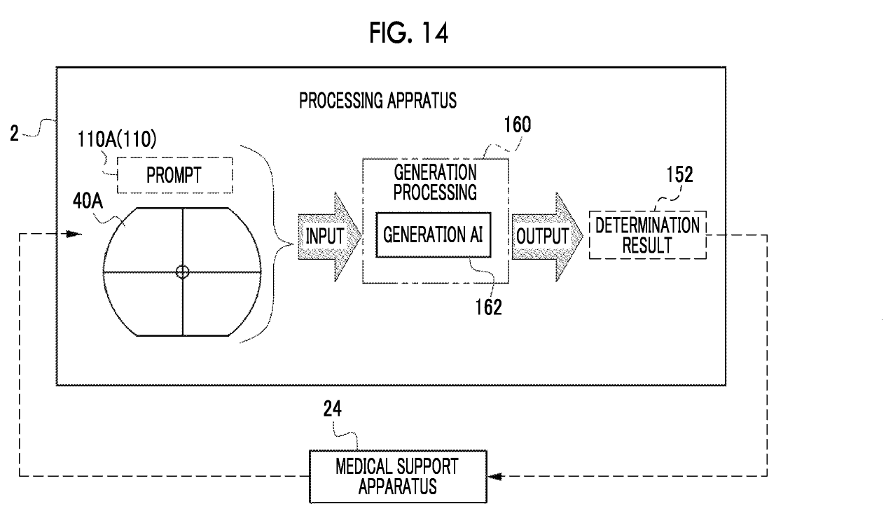

In the above description, the form example has been described in which the processing device 2 performs the recognition processing 112, the defect presence and absence specification processing 114, the type identification processing 116, the noise removal processing 140, and the edge extraction processing 142, but this is merely an example. For example, as shown in FIG. 14, the processing device 2 may perform generation processing 160 instead of the recognition processing 112, the defect presence and absence specification processing 114, the type identification processing 116, the noise removal processing 140, and the edge extraction processing 142.

In the generation processing 160, a generative AI 162 is used. Examples of the generative AI 162 include ChatGPT using GPT-4 (Internet search <https://openai.com/gpt-4>). The prompt 110A (for example, a prompt for instructing the generation of the determination result 152) that is the instruction data and the diagnostic frame 40A may be input to the generative AI 162 as the request information 110 transmitted from the medical support device 24, and the determination result 152 may be generated by the generative AI 162. The determination result 152 is transmitted to the medical support device 24 by the processing device 2 by the generative AI 162.

In addition, the information generated by the generative AI 162 is not limited to the determination result 152, and may be, for example, information (that is, the defect absence message 134, the defect presence message 136, and/or the bounding box BB, which are examples of a "diagnosis result" according to the present disclosure) displayed on the screen 35 shown in FIG. 9, or may be information obtained by converting the defect absence message 134 and the defect presence message 136 into voice. The information obtained by converting the defect absence message 134 and the defect presence message 136 into voice is an example of a "diagnosis result" according to the present disclosure.

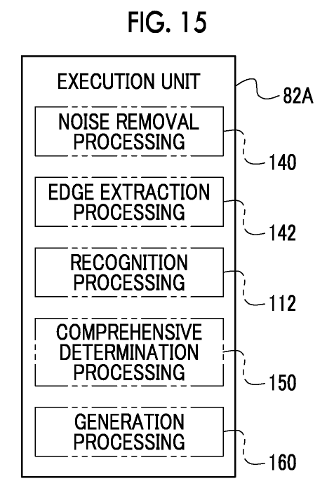

In the above-described embodiment, the form example (that is, the form example in which the execution unit 82A indirectly executes the recognition processing 112, the noise removal processing 140, the edge extraction processing 142, the comprehensive determination processing 150, and the generation processing 160 by using the processing device 2) in which the processing device 2 performs the recognition processing 112, the noise removal processing 140, the edge extraction processing 142, the comprehensive determination processing 150, and the generation processing 160 has been described, but the present disclosure is not limited thereto. For example, as shown in FIG. 15, the execution unit 82A may directly execute the recognition processing 112, the noise removal processing 140, the edge extraction processing 142, the comprehensive determination processing 150, and/or the generation processing 160.

In the above-described embodiment, the form example has been described in which the controller 82B generates the information obtained by visualizing the defect absence information 122 and displays the generated information on the screen 35, and generates the information obtained by visualizing the defect presence information 128 and displays the generated information on the screen 35, but the present disclosure is not limited thereto. For example, the processing device 2 may generate the information obtained by visualizing the defect absence information 122 and/or the defect presence information 128 and transmit the generated information to the medical support device 24 or the like, and the medical support device 24 or the like may display the information obtained by visualizing the defect absence information 122 and/or the defect presence information 128 on the screen 35 or the like by the processing device 2.

In the above-described embodiment, the form example has been described in which the diagnostic frame 40A generated by imaging the rear surface 98 on which the test chart is formed by the camera 52 is used for the diagnosis of the optical system 55, but this is merely an example, and the diagnostic frame 40A generated by imaging the rear surface 98 on which the test chart is not formed by the camera 52 may be used for the diagnosis of the optical system 55. The rear surface 98 on which the test chart is not formed refers to a monochromatic surface. Examples of the monochromatic surface include a white surface.

In the above-described embodiment, the form example has been described in which the information obtained by visualizing the defect absence information 122 and the defect presence information 128 is displayed on the screen 35 of the display device 18, but this is merely an example, and the defect absence information 122 and/or the defect presence information 128 may be displayed in a distributed manner on a plurality of display devices.

In the above-described embodiment, the recognition processing 112 using AI of the bounding box method is described as an example, but this is merely an example, and, for example, recognition processing using AI of a segmentation method may be executed instead of the recognition processing 112 using AI of the bounding box method.

In the embodiment described above, the form example has been described in which the diagnosis support processing is performed by the computer 78, but the present disclosure is not limited to this. At least a part of processing included in the diagnosis support processing may be performed by a device provided outside the computer 78.

In the embodiment described above, although a form example has been described in which the processing device 2 is realized by cloud computing, this is merely an example. The processing device 2 may be realized by network computing such as fog computing, edge computing, or grid computing.

In the above-described embodiment, the form example has been described in which the diagnostic program 108 is stored in the storage 86, but the present disclosure is not limited to this. For example, the diagnostic program 108 may be stored in a portable computer-readable non-transitory storage medium, such as an SSD or a USB memory. The diagnostic program 108 stored in the non-transitory storage medium is installed in the computer 78 of the endoscope apparatus 10. The processor 82 executes the diagnosis processing in accordance with the diagnostic program 108.

In addition, the diagnostic program 108 may be stored in a storage device of another computer, a server, or the like connected to the endoscope apparatus 10 via a network, and the diagnostic program 108 may be downloaded in response to a request from the endoscope apparatus 10 and installed in the computer 78.

It is not necessary to store all of the diagnostic program 108 in the storage device of another computer, a server apparatus, or the like connected to the endoscope apparatus 10, or to store all of the diagnostic program 108 in the storage 86, and a part of the diagnostic program 108 may be stored.

The following various processors can be used as a hardware resource for executing the information processing. Examples of the processor include a CPU that is a general-purpose processor functioning as the hardware resource for executing the processing by executing software, that is, a program. In addition, examples of the processor include a dedicated electric circuit which is a processor having a circuit configuration designed to be dedicated to performing specific processing, such as an FPGA, a PLD, or an ASIC. Any processor has a memory built into or connected to it, and any processor uses the memory to execute processing.

The hardware resource for executing the diagnosis support processing may be configured by one of the various processors or by a combination of two or more processors of the same type or different types (for example, a combination of a plurality of FPGAs or a combination of a CPU and an FPGA). In addition, the hardware resource for executing the diagnosis processing may be one processor.

A first example in which the hardware resource is composed of one processor is an aspect in which one or more CPUs and software are combined to constitute one processor and the processor functions as the hardware resource that executes the processing. As a second example, as typified by a SoC or the like, there is a form in which a processor that implements all functions of a system including a plurality of hardware resources executing the diagnosis support processing with one IC chip is used. As described above, the diagnosis support processing is implemented using one or more of the various processors as the hardware resource.

Further, as a hardware structure of the various processors, more specifically, an electric circuit obtained by combining circuit elements such as semiconductor elements can be used. In addition, the diagnosis processing is merely an example. Therefore, needless to say, unnecessary steps may be deleted, new steps may be added, or the processing order may be changed without departing from the spirit and scope of the present disclosure.

The contents described and shown above are detailed descriptions of parts related to the present disclosure and are merely examples of the present disclosure. For example, description related to the above configurations, functions, actions, and effects is description related to an example of configurations, functions, actions, and effects of the parts relating to the present disclosure. Therefore, it goes without saying that unnecessary parts may be deleted, new elements may be added, or replacements may be made with respect to the above-described contents and the above-shown contents within a range that does not deviate from the gist of the present disclosure. Further, in order to avoid complications and to easily understand the portions according to the present disclosure, in the content described and illustrated above, common technical knowledge and the like that do not need to be described to implement the present disclosure are not described.

All of the documents, the patent applications, and the technical standards described in the present specification are incorporated into the present specification by reference to the same extent as in a case in which each of the documents, the patent applications, and the technical standards are specifically and individually stated to be described by reference.

1. . A diagnostic apparatus comprising:
a processor that is used for an endoscope having a distal end portion configured to irradiate an inside of a body with light, the distal end portion being provided with an optical system of a camera configured to image the inside of the body,
wherein the processor is configured to:
execute a diagnosis of the optical system by performing image analysis using AI on a rear surface image obtained by imaging, by the camera, a rear surface in a state in which the rear surface is irradiated with the light in a situation in which a cap that is mounted on the distal end portion to cover the optical system, the cap having a rear surface configured to reflect the light, is mounted on the distal end portion; and
output a diagnostic result obtained by executing the diagnosis.

2. . The diagnostic apparatus according to claim 1,
wherein the diagnosis includes defect presence and absence identification processing of identifying presence or absence of a defect of the optical system.

3. . The diagnostic apparatus according to claim 1,
wherein the diagnosis includes type identification processing of identifying a type of a defect of the optical system, and
the type of the defect includes a first type that does not require collection of the optical system and a second type that requires the collection of the optical system.

4. . The diagnostic apparatus according to claim 3,
wherein the type identification processing includes a classification processing of classifying the type of the defect into the first type and the second type.

5. . The diagnostic apparatus according to claim 3,
wherein the first type is contamination of the optical system, and
the second type is a failure of the optical system.

6. . The diagnostic apparatus according to claim 1,
wherein the image analysis is implemented by inputting the rear surface image to a trained model that generates information assuming the diagnostic result or information serving as a basis for a diagnosis result by inputting information assuming the rear surface image, to generate the diagnosis result or the information serving as the basis for the diagnosis result.

7. . The diagnostic apparatus according to claim 3,
wherein the AI includes a first type identification AI that identifies the first type based on the rear surface image, and a second type identification AI that identifies the second type based on the rear surface image.

8. . The diagnostic apparatus according to claim 1,
wherein the AI is enhanced by performing retraining based on the diagnostic result.

9. . The diagnostic apparatus according to claim 1,
wherein the diagnosis includes position identification processing of identifying a position at which a defect of the optical system occurs.

10. . The diagnostic apparatus according to claim 1,
wherein the rear surface image is an image obtained by performing noise removal processing and/or edge extraction processing.

11. . The diagnostic apparatus according to claim 10,
wherein the rear surface image is an image obtained by performing the edge extraction processing after performing the noise removal processing.

12. . The diagnostic apparatus according to claim 1,
wherein the rear surface has a first region that reflects the light and a second region that has a lower reflectance than the first region.

13. . The diagnostic apparatus according to claim 12,
wherein an area of the first region is larger than an area of the second region, and a spatial frequency of the second region is higher than a spatial frequency of the first region.

14. . The diagnostic apparatus according to claim 12,
wherein the second region is a test chart.

15. . The diagnostic apparatus according to claim 1,
wherein the rear surface is formed in a curved shape.

16. . The diagnostic apparatus according to claim 1,
wherein the rear surface is a surface that diffuses the light.

17. . The diagnostic apparatus according to claim 1,
wherein the cap is formed in a two-layer structure.

18. . The diagnostic apparatus according to claim 17,
wherein the two-layer structure is formed of an inner layer and an outer layer, and a hollow region is provided between the inner layer and the outer layer.

19. . The diagnostic apparatus according to claim 1,
wherein a color of an outer surface of the cap is black.

20. . The diagnostic apparatus according to claim 1,
wherein the cap is mounted on the distal end portion via an attachment, and
a diameter of a portion of the attachment that connects the cap and the distal end portion is adjustable.

21. . A diagnostic system comprising:
a terminal that is used for an endoscope having a distal end portion configured to irradiate an inside of a body with light, the distal end portion being provided with an optical system of a camera configured to image an inside of the body; and
a server,
wherein the terminal is configured to transmit, to the server, a rear surface image obtained by imaging, by the camera, a rear surface in a state in which the rear surface is irradiated with the light in a situation in which a cap that is mounted on the distal end portion to cover the optical system, the cap having a rear surface configured to reflect the light is mounted on the distal end portion,
the server is configured to:
execute a diagnosis of the optical system by performing image analysis using AI on a rear surface image; and
transmit a diagnosis result obtained by executing the diagnosis to the terminal, and
the terminal receives the diagnostic result.

22. . A diagnostic method that is used for an endoscope having a distal end portion configured to irradiate an inside of a body with light, the distal end portion being provided with an optical system of a camera configured to image the inside of the body, the diagnostic method comprising:
executing a diagnosis of the optical system by performing image analysis using AI on a rear surface image obtained by imaging, by the camera, a rear surface in a state in which the rear surface is irradiated with the light in a situation in which a cap that is mounted on the distal end portion to cover the optical system, the cap having a rear surface configured to reflect the light is mounted on the distal end portion; and outputting a diagnostic result obtained by executing the diagnosis.

23. . A non-transitory computer-readable storage medium storing a program executable by a computer that is used for an endoscope having a distal end portion configured to irradiate an inside of a body with light and having a distal end portion provided with an optical system of a camera configured to image the inside of the body to execute a process comprising:
executing a diagnosis of the optical system by performing image analysis using AI on a rear surface image obtained by imaging, by the camera, a rear surface in a state in which the rear surface is irradiated with the light in a situation in which a cap that is mounted on the distal end portion to cover the optical system, the cap having a rear surface configured to reflect the light is mounted on the distal end portion; and outputting a diagnostic result obtained by executing the diagnosis.
